(19) United States
(12) Patent Application Publication
(10) Pub. No.: US 2010/0249234 A1
(43) Pub. Date: Sep. 30, 2010

(54) METHODS OF REDUCING VIRULENCE IN BACTERIA

(75) Inventors: Ching-Hong Yang, Mequon, WI (US); Shihui Yang, Knoxville, TN (US)

Correspondence Address: MICHAEL BEST & FRIEDRICH LLP, 100 E WISCONSIN AVENUE, Suite 3300, MILWAUKEE, WI, 53202, US

(73) Assignee: UWM RESEARCH FOUNDATION, INC., Milwaukee, WI (US)

(21) Appl. No.: 12/595,148

(22) PCT Filed: Apr. 10, 2008
(86) PCT No.: PCT/US08/59928

§ 371 Date: Oct. 8, 2009

Related U.S. Application Data
(63) Continuation of application No. 61/013,440, filed on Dec. 13, 2007.
(60) Provisional application No. 60/910,865, filed on Apr. 10, 2007.

(51) Int. Cl. A61K 31/192; C12Q 1/00; C12Q 1/68; C12Q 1/34; C12Q 1/37; A61K 31/045; C12Q 1/527; A01N 37/10; A01N 31/04

(52) U.S. Cl. 514/570; 435/4; 435/6; 435/18; 435/23; 514/730

(57) ABSTRACT

A method of reducing virulence in a bacterium comprising at least one of a GacS/GacA-type system, a HrpX/HrpY-type system, a T3SS-type system, and a Rsm-type system, the method comprising contacting the bacterium with an effective amount of a phenylpropanoid-type inhibitory compound.

### CROSS-REFERENCE TO RELATED APPLICATIONS

This application claims priority to U.S. Provisional Applications No. 60/910,865, filed Apr. 10, 2007, and 61/013,440, filed Dec. 13, 2007, each of which is incorporated herein by reference in its entirety.

### STATEMENT REGARDING FEDERALLY SPONSORED RESEARCH OR DEVELOPMENT

This invention was made with government support under EF-0332163 awarded by the National Science Foundation. The United States government has certain rights in the invention.

### FIELD OF THE INVENTION

The present invention relates to methods and compounds for controlling virulence in bacteria, methods of identifying further compounds for controlling virulence in bacteria, and methods, compounds, and compositions for treating subjects with bacterial infections to reduce virulence of bacteria in said subjects.

### BACKGROUND

GacS/GacA is a two component signal transduction system (“TCSTS”) that is widely distributed in many bacteria to respond to environmental stimuli and adapt to different environmental conditions. GacS is a putative histidine kinase sensor and GacA is the response regulator. The homologs of the TCSTS of GacS/GacA have been reported in a variety of Gram-negative bacteria, including E. coli (BarA/UvrY), Pectobacterium spp., S. typhimurium (BarA/SirA), Pseudomonas spp. (GacS/GacA), and Legionella pneumophila (LetS/LetA), Vibrio species.

GacS/GacA and homologous systems regulate many virulence factors including, but not limited to, regulatory RNA, quorum sensing (“QS”) signals, type III secretion system (“T3SS”) genes, pectate lyases, proteases, biofilm formation, and toxins. For example, in D. dadantii, the GacS/GacA system is located at the top of a regulatory cascade and functions as a central regulator by controlling an assortment of transcriptional and posttranscriptional factors. Mainly, the influence of the GacS/GacA system on pectinase production and T3SS gene expression is channeled through a regulatory RNA system, the Rsm system, by activating rsmB which binds to and inhibits the T3SS mRNA decay effect of RsmA.

In addition to the GacS/GacA-RsmA-rsmB-hrpL regulatory pathway, the T3SS of Dickeya dadantii, which belongs to Group I T3SS of phytobacteria, is regulated by a HrpX/Y-HrpS-HrpL pathway. The two-component system HrpX/HrpY activates the gene encoding HrpS, which is required for expression of hrpL. HrpL, an alternative sigma factor, further activates expression of genes encoding the T3SS apparatus and its secreted products. Thus, the GacS/GacA and HrpX/HrpY TCSTS systems both exert a regulatory effect in D. dadantii, in particular through the T3SS system.

### SUMMARY OF THE INVENTION

In one embodiment, the invention provides a method of reducing virulence in a bacterium comprising at least one of a GacS/GacA-type system, a HrpX/HrpY-type system, a T3SS-type system, and a Rsm-type system. The method comprises contacting the bacterium with an effective amount of a phenylpropanoid-type inhibitory compound.

In another embodiment, the invention provides a method of treating a subject having a bacterium associated therewith, the bacterium comprising at least one of a GacS/GacA-type system, a HrpX/HrpY-type system, a T3SS-type system, and a Rsm-type system. The method comprises administering to the subject an effective amount of a composition comprising a phenylpropanoid-type inhibitory compound.

In yet another embodiment, the invention provides a method of reducing virulence of a bacterium on a surface comprising a bacterium having at least one of a GacS/GacA-type system, a HrpX/HrpY-type system, a T3SS-type system, and a Rsm-type system. The method comprises contacting the surface with an effective amount of a composition comprising a phenylpropanoid-type inhibitory compound.

In another embodiment, the invention is a pharmaceutical composition comprising a phenylpropanoid-type inhibitory compound according to formula (II) and a pharmaceutically-acceptable carrier or diluent:

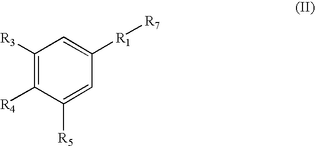

wherein R₁ is an alkylene;

R₃ and R₅ are hydrogen, R₄ is hydrogen, hydroxy, sulfhydryl or halo, and R₇ is hydroxy, carboxy or formyl;

R₃, R₄, and R₅ are independently selected from hydrogen, alkyl, alkenyl, alkynyl, cycloalkyl, heterocyclyl, aryl, heteroaryl, halo, hydroxy, ether, alkoxy, acetal, hemiacetal, ketal, hemiketal, formyl, acyl, carboxy, thiocarboxy, thiolcarboxy, thionocarboxy, imidic acid, hydroxamic acid, ester, acyloxy, oxycarboyloxy, amino, amido, thioamido, acylamido, aminocarbonyloxy, ureido, guanidine, amindino, nitro, nitroso, azido, cyano, isocyano, isocyanato, thiocyano, isothiocyano, sulfhydryl, thioether, disulfide, sulfine, sulfonyl, sulfinic acid, sulfonic acid, sulfinate, sulfonate, sulfinyloxy, sulfonyloxy, sulfate, sulfamyl, sulfonamide, sulfamino, sulfonamino, sulfinamino, phosphino, phosphor, phosphinyl, phosphono, phosphonate, phosphonooxy, phosphate, phosphorous acid, phosphite, phosphoramidite, or phosphoramidate, wherein two of R₃, R₄, and R₅ optionally are linked together to form a ring; and

R₇ is hydroxy, acetal, hemiacetal, ketal, hemiketal, formyl, acyl, carboxy, thiocarboxy, thiolcarboxy, thionocarboxy, imidic acid, hydroxamic acid, ester, acyloxy, oxycarboyloxy, amino, amido, thioamido, acylamido, aminocarbonyloxy, ureido, guanidine, amindino, nitro, nitroso, azido, cyano, isocyano, isocyanato, thiocyano, isothiocyano, sulfhydryl, thioether, disulfide, sulfine, sulfonyl, sulfinic acid, sulfonic acid, sulfinate, sulfonate, sulfinyloxy, sulfonyloxy, sulfate, sulfamyl, sulfonamide, sulfamino, sulfonamino, sulfinamino, phosphino, phosphor, phosphinyl, phosphono, phosphonate, phosphonooxy, phosphate, phosphorous acid, phosphite, phosphoramidite, or phosphoramidate.

In still another embodiment, the invention is a method of screening a compound for an ability to reduce virulence of a bacterium comprising at least one of a GacS/GacA-type system, a HrpX/HrpY-type system, a T3SS-type system, and a Rsm-type system. The method comprises contacting the bacterium with a phenylpropanoid derivative and detecting at least one of: (i) a change in a component of at least one of the GacS/GacA-type system, the HrpX/HrpY-type system, the T3SS-type system, and the Rsm-type system of the bacterium, and (ii) a change in host pathology.

### BRIEF DESCRIPTION OF THE DRAWINGS

FIG. 1 shows a regulatory network of type III secretion system (T3SS) regulatory pathways of Dickeya dadantii 3937 (Ech3937).

FIGS. 2A and 2B show the promoter activities of hrpN in Dickeya dadantii 3937 (Ech3937) using FACS analysis at 12 h (FIG. 2A) and 24 h (FIG. 2B).

FIG. 3 shows the promoter activities of hrpA, hrpN, hrpL, and hrpS in Dickeya dadantii 3937 (Ech3937) and hrpL mutant WPP96 using FACS analysis.

FIG. 4 shows the relative mRNA level of hrpY, hrpS, hrpL, dspE, hrpA, rsmB, and gacA of Dickeya dadantii 3937 (Ech3937) using quantitative RT-PCR (qRT-PCR).

FIG. 5 shows the promoter activity of hrpN of Dickeya dadantii 3937 (Ech3937), hrpS mutant WPP90, hrpX mutant WPP67, hrpY mutant WPP92, and Ech3937 (pAT) using FACS analysis.

FIG. 6A shows biofilm and pellicle formation in SOBG broth.

FIG. 6B shows cross sections of the pellicle observed with scanning electron microscopy at different magnifications.

FIG. 7 shows pectate lyase (Pel), protease (Prt), and cellulase (Cel) production of wild-type Ech-Rif, gacA mutant Ech137, and gacA mutant complemented strain Ech137 (pCLgacA) examined by plate assays.

FIG. 8 shows spectrophotometric quantification of pectate lyase (Pel) activity for Ech-Rif, gacA mutant Ech137, and the complementary strain Ech137 (pCLgacA).

FIG. 9 shows promoter activity of pelD and pelL in Ech-Rif (black line with black filling) and gacA mutant Ech137 (gray line).

FIG. 10A shows relative levels of rsmA, rsmB, rsmC, and hrpL mRNA in gacA mutant Ech137 compared with wild-type Ech-Rif grown for 6 or 12 h in a minimal medium.

FIG. 10B shows relative levels of gacA and rsmB mRNA in gacA mutant Ech137 and gacA mutant complemented strain Ech137 (pCLgacA) compared with wild-type Ech-Rif grown for 12 h in a minimal medium.

FIG. 11 shows local maceration lesions caused by a, Ech-Rif; b, gacA mutant Ech137; and c, complemented strain Ech137 (pCLgacA).

FIG. 12 shows the bacterial population and pectinase activity of Ech-Rif (solid diamonds) and Ech137 (solid triangles) determined by plate assay and spectrophotometric quantification, respectively.

FIG. 13 shows the development of systemic symptoms caused by Ech-Rif and Ech137 strains in African violet cv. Gauguin (Saintpaulia ionantha) plants.

FIG. 14 shows the relative mRNA level of hrpS, hrpL, dspE, hrpA, hrpN, and rsmB of Dickeya dadantii 3937 (Ech3937) in minimum medium (MM) supplemented with 0.1 mM p-coumaric acid (PCA) compared to those in MM without PCA.

FIG. 15 shows HrpN protein expression of Dickeya dadantii 3937 (Ech3937) in minimum medium (MM) and MM supplemented with p-Coumaric acid (PCA).

FIGS. 16A and 16B show the promoter activities of hrpA in Dickeya dadantii 3937 (Ech3937) grown in minimum medium (MM) and MM supplemented with different amount of p-coumaric acid (PCA) at 12 h (FIG. 16A) and 24 h (FIG. 16B).

FIG. 17 shows pectate lyase (Pel) production of Dickeya dadantii 3937 (Ech3937) grown in minimal medium (MM) and MM supplemented with 0.1 mM of p-coumaric acid (PCA) at 12 h.

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limited.

### DETAILED DESCRIPTION OF THE INVENTION

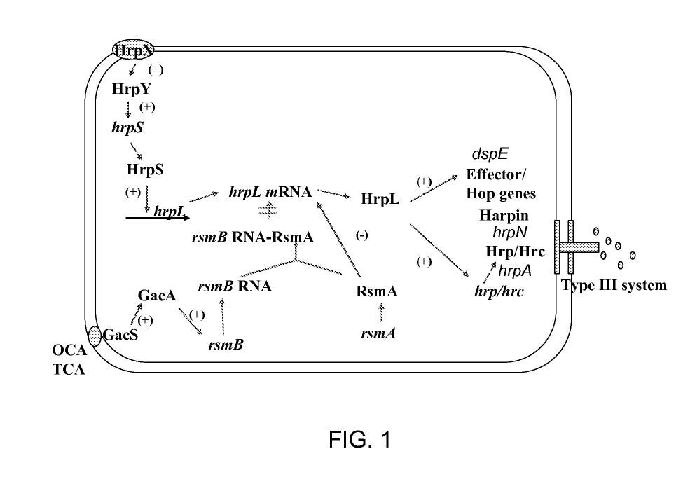

A regulatory role for the two-component system GacS/GacA on the type III secretion system (“T3SS”) of Dickeya dadantii 3937 (Ech3937) has been demonstrated to channel through a regulator of secondary metabolism (Rsm) system. Rsm is a novel type of post-transcriptional regulatory system that plays a critical role in gene expression. RsmA is a small RNA-binding protein that acts by lowering the half-life of the target mRNA. rsmB is an untranslated regulatory RNA that binds RsmA and inhibits its activity by forming an inactive ribonucleoprotein complex. In Ech3937, GacS/GacA upregulates hrpL mRNA through a post-transcriptional regulation by enhancing the rsmB RNA level, which binds to RsmA and inhibits the hrpL mRNA decay effect of RsmA (FIG. 1).

FIG. 1 shows a regulatory network of type III secretion system (T3SS) of Dickeya dadantii 3937 (Ech3937). Lines with a (+) symbol designate positive regulation, and the line with a (−) symbol indicates negative regulation. The T3SS of Ech3937 is regulated by the HrpX/HrpY-HrpS-HrpL and GacS/GacA-rsmB-HrpL regulatory pathways. The two-component system HrpX/HrpY activates the gene encoding a σ54-enhancer HrpS, which is required for expression of an alternative sigma factor, hrpL. HrpL further activates expression of genes encoding the T3SS apparatus and its secreted products.

The T3SS contributes to bacterial virulence within a host. A gacA deletion mutant of Dickeya dadantii (Erwinia chrysanthemi 3937) was found to exhibit diminished production of pectate lyase, protease, and cellulose, enzymes that normally lead to loss of structural integrity of plant cell walls. Diminished production of enzymes that attack the plant cell walls leads to diminished bacterial virulence.

Several compounds, including o-coumaric acid (“OCA”) and t-cinnamic acid (“TCA”), have been identified as inducers of the GacS/GacA regulatory system. Induction of the GacS/GacA system in turn affects the Rsm system, which further affects the expression of T3SS genes, including pectinase genes.

In further studies the inventors have screened compounds, including t-cinnamic acid, o-coumaric acid, m-coumaric acid, p-coumaric acid, hydrocinnamic acid, phenoxyacetic acid, trans-2-phenylcyclopropane-1-carboxylic acid, trans-3-(3-pyridyl)acrylic acid, trans-3-indoleacrylic acid, 2-methylcinnamic acid, 2-chlorocinnamic acid, methyl trans-cinnamate, and cinnamyl alcohol. Two compounds, p-coumaric acid (PCA) and cinnamyl alcohol, were found to reduce induction of virulence in Dickeya dadantii. Without being limited as to theory, PCA appears to reduce virulence through the HrpX/HrpY system (see FIG. 1, Example 10).

Based on the discovery that OCA, TCA, PCA, and cinnamyl alcohol can regulate (i.e. promote or reduce) bacterial virulence, further screening will be conducted in order to identify compounds that reduce bacterial virulence. Various compounds will be synthesized and screened for their ability to reduce bacterial virulence. The synthesized compounds will be tested for an ability to reduce virulence of bacteria having a two-component signal transduction system such as a GacS/GacA system (or a homolog of the GacS/GacA system) or a HrpX/HrpY system (or a homolog of the HrpX/HrpY system), an Rsm system (or homolog), and/or a T3SS system (or homolog).

The compounds that will be produced for possible testing are referred to herein as “phenylpropanoid derivatives.” Phenylpropanoids are plant-derived organic compounds synthesized from phenylalanine Phenylpropanoid derivatives can be made by adding or removing substituents using methods known to those of skill in the art. Phenylpropanoid derivatives such as those disclosed herein can be synthesized de novo or by modifying naturally-occurring compounds.

A phenylpropanoid derivative may be a compound of formula (I).

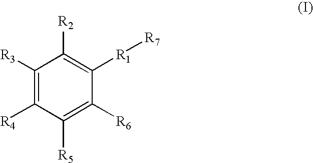

wherein R₁ is an alkylene;

R₂, R₃, R₄, R₅ and R₆ are independently selected from hydrogen, alkyl, alkenyl, alkynyl, cycloalkyl, heterocyclyl, aryl, heteroaryl, halo, hydroxy, ether, alkoxy, acetal, hemiacetal, ketal, hemiketal, formyl, acyl, carboxy, thiocarboxy, thiolcarboxy, thionocarboxy, imidic acid, hydroxamic acid, ester, acyloxy, oxycarboyloxy, amino, amido, thioamido, acylamido, aminocarbonyloxy, ureido, guanidine, amindino, nitro, nitroso, azido, cyano, isocyano, isocyanato, thiocyano, isothiocyano, sulfhydryl, thioether, disulfide, sulfine, sulfonyl, sulfinic acid, sulfonic acid, sulfinate, sulfonate, sulfinyloxy, sulfonyloxy, sulfate, sulfamyl, sulfonamide, sulfamino, sulfonamino, sulfinamino, phosphino, phosphor, phosphinyl, phosphono, phosphonate, phosphonooxy, phosphate, phosphorous acid, phosphite, phosphoramidite, or phosphoramidate, wherein two of R₂, R₃, R₄, R₅, and R₆ optionally are linked together to form a ring;

and

R₇ is hydroxy, acetal, hemiacetal, ketal, hemiketal, formyl, acyl, carboxy, thiocarboxy, thiolcarboxy, thionocarboxy, imidic acid, hydroxamic acid, ester, acyloxy, oxycarboyloxy, amino, amido, thioamido, acylamido, aminocarbonyloxy, ureido, guanidine, amindino, nitro, nitroso, azido, cyano, isocyano, isocyanato, thiocyano, isothiocyano, sulfhydryl, thioether, disulfide, sulfine, sulfonyl, sulfinic acid, sulfonic acid, sulfinate, sulfonate, sulfinyloxy, sulfonyloxy, sulfate, sulfamyl, sulfonamide, sulfamino, sulfonamino, sulfinamino, phosphino, phosphor, phosphinyl, phosphono, phosphonate, phosphonooxy, phosphate, phosphorous acid, phosphite, phosphoramidite, or phosphoramidate.

### DEFINITIONS

Each of the groups defined below may be substituted with one or more of the moieties defined herein.

Alkyl: The term “alkyl” as used herein, pertains to a monovalent moiety obtained by removing a hydrogen atom from a carbon atom of a hydrocarbon compound having from 1 to 20 carbon atoms (unless otherwise specified), which may be aliphatic or alicyclic, and which may be saturated or unsaturated (e.g., partially unsaturated, fully unsaturated). Thus, the term “alkyl” includes the sub-classes alkenyl, alkynyl, cycloalkyl, cycloalkyenyl, cylcoalkynyl, etc., discussed below.

In the context of alkyl groups, the prefixes (e.g., C₁₋₄, C₁₋₇, C₁₋₂₀, C₂₋₇, C₃₋₇, etc.) denote the number of carbon atoms, or range of number of carbon atoms. For example, the term “C₁₋₄ alkyl” as used herein, pertains to an alkyl group having from 1 to 4 carbon atoms. Examples of groups of alkyl groups include C₁₋₄ alkyl (“lower alkyl”), C₁₋₇ alkyl, C₁₋₂₀ alkyl and C₁₋₃₀ alkyl. Note that the first prefix may vary according to other limitations; for example, for unsaturated alkyl groups, the first prefix must be at least 2; for cyclic and branched alkyl groups, the first prefix must be at least 3; etc.

Examples of saturated alkyl groups include, but are not limited to, methyl (C₁), ethyl (C₂), propyl (C₃), butyl (C₄), pentyl (C₅), hexyl (C₆), heptyl (C₇), octyl (C₈), nonyl (C₉), decyl (C₁₀), undecyl (C₁₁), dodecyl (C₁₂), tridecyl (C₁₃), tetradecyl (C₁₄), pentadecyl (C₁₅), and eicodecyl (C₂₀).

Examples of saturated linear alkyl groups include, but are not limited to, methyl (C₁), ethyl (C₂), n-propyl (C₃), n-butyl (C₄), n-pentyl (amyl) (C₅), n-hexyl (C₆), and n-heptyl (C₇).

Examples of saturated branched alkyl groups include iso-propyl (C₃), iso-butyl (C₄), sec-butyl (C₄), tert-butyl (C₄), iso-pentyl (C₅), and neo-pentyl (C₅).

Alkenyl: The term “alkenyl” as used herein, pertains to an alkyl group having one or more carbon-carbon double bonds. Examples of groups of alkenyl groups include C₂₋₄ alkenyl, C₂₋₇ alkenyl, C₂₋₂₀ alkenyl.

Examples of unsaturated alkenyl groups include, but are not limited to, ethenyl (vinyl, —CH═CH₂), 1-propenyl (—CH═CH—CH₃), 2-propenyl (allyl, —CH—CH═CH₂), isopropenyl (1-methylvinyl, —C(CH₃)═CH₂), butenyl (C₄), pentenyl (C₅), and hexenyl (C₆).

Alkynyl: The term “alkynyl” as used herein, pertains to an alkyl group having one or more carbon-carbon triple bonds. Examples of groups of alkynyl groups include C₂₋₄ alkynyl, C₂₋₇ alkynyl, C₂₋₂₀ alkynyl.

Examples of unsaturated alkynyl groups include, but are not limited to, ethynyl (ethinyl, —C≡CH) and 2-propynyl (propargyl, —CH₂—C≡CH).

Cycloalkyl: The term “cycloalkyl” as used herein, pertains to an alkyl group which is also a cyclyl group; that is, a monovalent moiety obtained by removing a hydrogen atom from an alicyclic ring atom of a carbocyclic ring of a carbocyclic compound, which carbocyclic ring may be saturated or unsaturated (e.g., partially unsaturated, fully unsaturated), which moiety has from 3 to 20 carbon atoms (unless otherwise specified), including from 3 to 20 ring atoms. Thus, the term “cycloalkyl” includes the sub-classes cycloalkyenyl and cycloalkynyl. Preferably, each ring has from 3 to 7 ring atoms. Examples of groups of cycloalkyl groups include C₃₋₃₀ cycloalkyl, C₃₋₂₀ cycloalkyl, C₃₋₁₀ cycloalkyl, C₃₋₇ cycloalkyl.

Examples of cycloalkyl groups include, but are not limited to, those derived from:

saturated monocyclic hydrocarbon compounds:

cyclopropane (C₃), cyclobutane (C₄), cyclopentane (C₅), cyclohexane (C₆), cycloheptane (C₇), methylcyclopropane (C₄), dimethylcyclopropane (C₅), methylcyclobutane (C₅), dimethylcyclobutane (C₆), methylcyclopentane (C₆), dimethylcyclopentane (C₇), methylcyclohexane (C₇), dimethylcyclohexane (C₈), menthane (C₁₀);

unsaturated monocyclic hydrocarbon compounds:

cyclopropene (C₃), cyclobutene (C₄), cyclopentene (C₅), cyclohexene (C₆), methylcyclopropene (C₄), dimethylcyclopropene (C₅), methylcyclobutene (C₅), dimethylcyclobutene (C₆), methylcyclopentene (C₆), dimethylcyclopentene (C₇), methylcyclohexene (C₇), dimethylcyclohexene (C₈);

saturated polycyclic hydrocarbon compounds:

thujane (C₁₀), carane (C₁₀), pinane (C₁₀), bornane (C₁₀), norcarane (C₇), norpinane (C₇), norbornane (C₇), adamantane (C₁₀), decalin (decahydronaphthalene) (C₁₀);

unsaturated polycyclic hydrocarbon compounds:

camphene (C₁₀), limonene (C₁₀), pinene (C₁₀);

polycyclic hydrocarbon compounds having an aromatic ring:

indene (C₉), indane (e.g., 2,3-dihydro-1H-indene) (C₉), tetraline (1,2,3,4-tetrahydronaphthalene) (C₁₀), acenaphthene (C₁₂), fluorene (C₁₃), phenalene (C₁₃), acephenanthrene (C₁₅), aceanthrene (C₁₆), cholanthrene (C₂₀).

Heterocyclyl: The term “heterocyclyl” as used herein, pertains to a monovalent moiety obtained by removing a hydrogen atom from a ring atom of a heterocyclic compound, which moiety has from 3 to 20 ring atoms (unless otherwise specified), of which from 1 to 10 are ring heteroatoms. Preferably, each ring has from 3 to 7 ring atoms, of which from 1 to 4 are ring heteroatoms.

In this context, the prefixes (e.g., C₃₋₂₀, C₃₋₇, C₅₋₆, etc.) denote the number of ring atoms, or range of number of ring atoms, whether carbon atoms or heteroatoms. For example, the term “C₅₋₆ heterocyclyl” as used herein, pertains to a heterocyclyl group having 5 or 6 ring atoms. Examples of groups of heterocyclyl groups include C₃₋₃₀ heterocyclyl, C₃₋₂₀ heterocyclyl, C₅₋₂₀ heterocyclyl, C₃₋₁₅ heterocyclyl, C₅₋₁₅ heterocyclyl, C₃₋₁₂ heterocyclyl, C₅₋₁₂ heterocyclyl, C₃₋₁₀ heterocyclyl, C₅₋₁₀ heterocyclyl, C₃₋₇ heterocyclyl, C₅₋₇ heterocyclyl, and C₅₋₆ heterocyclyl.

Examples of monocyclic heterocyclyl groups include, but are not limited to, those derived from:

N₁: aziridine (C₃), azetidine (C₄), pyrrolidine (tetrahydropyrrole) (C₅), pyrroline (e.g., 3-pyrroline, 2,5-dihydropyrrole) (C₅), 2H-pyrrole or 3H-pyrrole (isopyrrole, isoazole) (C₅), piperidine (C₆), dihydropyridine (C₆), tetrahydropyridine (C₆), azepine (C₇);

O₁: oxirane (C₃), oxetane (C₄), oxolane (tetrahydrofuran) (C₅), oxole (dihydrofuran) (C₅), oxane (tetrahydropyran) (C₆), dihydropyran (C₆), pyran (C₆), oxepin (C₇);

S₁: thiirane (C₃), thietane (C₄), thiolane (tetrahydrothiophene) (C₅), thiane (tetrahydrothiopyran) (C₆), thiepane (C₇);

O₂: dioxolane (C₅), dioxane (C₆), and dioxepane (C₇);

O₃: trioxane (C₆);

N₂: imidazolidine (C₅), pyrazolidine (diazolidine) (C₅), imidazoline (C₅), pyrazoline (dihydropyrazole) (C₅), piperazine (C₆);

N₁O₁: tetrahydrooxazole (C₅), dihydrooxazole (C₅), tetrahydroisoxazole (C₅), dihydroisoxazole (C₅), morpholine (C₆), tetrahydrooxazine (C₆), dihydrooxazine (C₆), oxazine (C₆);

N₁S₁: thiazoline (C₅), thiazolidine (C₅), thiomorpholine (C₆);

N₂O₁: oxadiazine (C₆);

O₁Sᵢ: oxathiole (C₅) and oxathiane (thioxane) (C₆); and,

N₁O₁S₁: oxathiazine (C₆).

Examples of substituted monocyclic heterocyclyl groups include those derived from saccharides, in cyclic form, for example, furanoses (C₅), such as arabinofuranose, lyxofuranose, ribofuranose, and xylofuranse, and pyranoses (C₆), such as allopyranose, altropyranose, glucopyranose, mannopyranose, gulopyranose, idopyranose, galactopyranose, and talopyranose.

Aryl: The term “aryl” as used herein, pertains to a monovalent moiety obtained by removing a hydrogen atom from an aromatic ring atom of an aromatic compound, which moiety has from 3 to 20 ring atoms (unless otherwise specified). Preferably, each ring has from 5 to 7 ring atoms.

In this context, the prefixes (e.g. C₃₋₂₀, C₅₋₇, C₅₋₆, etc.) denote the number of ring atoms, or range of number of ring atoms, whether carbon atoms or heteroatoms. For example, the term “C₅₋₆aryl” as used herein, pertains to an aryl group having 5 or 6 ring atoms. Examples of groups of aryl groups include C₃₋₃₀ aryl, C₃₋₂₀ aryl, C₅₋₂₀ aryl, C₅₋₁₅ aryl, C₅₋₁₂ aryl, C₅₋₁₀ aryl, C₅₋₇ aryl, C₅₋₆ aryl, C₅ aryl, and C₆ aryl.

The ring atoms may be all carbon atoms, as in “carboaryl groups”. Examples of carboaryl groups include C₃₋₂₀ carboaryl, C₅₋₂₀ carboaryl, C₅₋₁₅ carboaryl, C₅₋₁₂ carboaryl, C₅₋₁₀ carboaryl, C₅₋₇ carboaryl, C₅₋₆ carboaryl and C₆ carboaryl.

Examples of carboaryl groups include, but are not limited to, those derived from benzene (i.e., phenyl) (C₆), naphthalene (C₁₀), azulene (C₁₀), anthracene (C₁₄), phenanthrene (C₁₄), naphthacene (C₁₈), and pyrene (C₁₆).

Examples of aryl groups which comprise fused rings, at least one of which is an aromatic ring, include, but are not limited to, groups derived from indane (e.g., 2,3-dihydro-1H-indene) (C₉), indene (C₉), isoindene (C₉), tetraline (1,2,3,4-tetrahydronaphthalene (C₁₀), acenaphthene (C₁₂), fluorene (C₁₃), phenalene (C₁₃), acephenanthrene (C₁₅), and aceanthrene (C₁₆).

Alternatively, the ring atoms may include one or more heteroatoms, as in “heteroaryl groups”. Examples of heteroaryl groups include C₃₋₂₀ heteroaryl, C₅₋₂₀ heteroaryl, C₅₋₁₅ heteroaryl, C₅₋₁₂ heteroaryl, C₅₋₁₀ heteroaryl, C₅₋₇ heteroaryl, C₅₋₆ heteroaryl, C₅ heteroaryl, and C₆ heteroaryl.

Examples of monocyclic heteroaryl groups include, but are not limited to, those derived from:

N₁: pyrrole (azole) (C₅), pyridine (azine) (C₆);

O₁: furan (oxole) (C₅);

S₁: thiophene (thiole) (C₅);

N₁O₁: oxazole (C₅), isoxazole (C₅), isoxazine (C₆);

N₂O₁: oxadiazole (furazan) (C₅);

N₃O₁: oxatriazole (C₅);

N₁S₁: thiazole (C₅), isothiazole (C₅);

N₂: imidazole (1,3-diazole) (C₅), pyrazole (1,2-diazole) (C₅), pyridazine (1,2-diazine) (C₆), pyrimidine (1,3-diazine) (C₆) (e.g., cytosine, thymine, uracil), pyrazine (1,4-diazine) (C₆);

N₃: triazole (C₅), triazine (C₆); and,

N₄: tetrazole (C₅).

Examples of heteroaryl groups which comprise fused rings, include, but are not limited to:

C₉ heteroaryl groups (with 2 fused rings) derived from benzofuran (O₁), isobenzofuran (O₁), indole (N₁), isoindole (N₁), indolizine (N₁), indoline (N₁), isoindoline (N₁), purine (N₄) (e.g., adenine, guanine), benzimidazole (N₂), indazole (N₂), benzoxazole (N₁O₁), benzisoxazole (N₁O₁), benzodioxole (O₂), benzofurazan (N₂O₁), benzotriazole (N₃), benzothiofuran (S₁), benzothiazole (N₁S₁), benzothiadiazole (N₂S);

C₁₀ heteroaryl groups (with 2 fused rings) derived from chromene (O₁), isochromene (O₁), chroman (O₁), isochroman (O₁), benzodioxan (O₂), quinoline (N₁), isoquinoline (N₁), quinolizine (N₁), benzoxazine (N₁O₁), benzodiazine (N₂), pyridopyridine (N₂), quinoxaline (N₂), quinazoline (N₂), cinnoline (N₂), phthalazine (N₂), naphthyridine (N₂), pteridine (N₄);

C₁₁ heteroaryl groups (with 2 fused rings) derived from benzodiazepine (N₂);

C₁₃ heteroaryl groups (with 3 fused rings) derived from carbazole (N₁), dibenzofuran (O₁), dibenzothiophene (S₁), carboline (N₂), perimidine (N₂), pyridoindole (N₂); and,

C₁₄ heteroaryl groups (with 3 fused rings) derived from acridine (N₁), xanthene (O₁), thioxanthene (S₁), oxanthrene (O₂), phenoxathiin (O₁S₁), phenazine (N₂), phenoxazine (N₁O₁), phenothiazine (N₁S₁), thianthrene (S₂), phenanthridine (N₁), phenanthroline (N₂), phenazine (N₂).

Heteroaryl groups which have a nitrogen ring atom in the form of an —NH— group may be N-substituted, that is, as —NR—. For example, pyrrole may be N-methyl substituted, to give N-methylpyrrole. Examples of N-substitutents include, but are not limited to C₁₋₇ alkyl, C₃₋₂₀ heterocyclyl, C₅₋₂₀ aryl, and acyl groups.

Heterocyclic groups (including heteroaryl groups) which have a nitrogen ring atom in the form of an —N=group may be substituted in the form of an N-oxide, that is, as —N(→O)═ (also denoted —N⁺(→O⁻)═). For example, quinoline may be substituted to give quinoline N-oxide; pyridine to give pyridine N-oxide; benzofurazan to give benzofurazan N-oxide (also known as benzofuroxan).

Cyclic groups may additionally bear one or more oxo (═O) groups on ring carbon atoms.

The above groups, whether alone or part of another substituent, may themselves optionally be substituted with one or more groups selected from themselves and the additional substituents listed below.

Halo: —F, —Cl, —Br, and —I.

Hydroxy: —OH.

Ether: —OR, wherein R is an ether substituent, for example, a C₁₋₇ alkyl group (also referred to as a C₁₋₇ alkoxy group, discussed below), a C₃₋₂₀ heterocyclyl group (also referred to as a C₃₋₂₀ heterocyclyloxy group), or a C₅₋₂₀ aryl group (also referred to as a C₅₋₂₀ aryloxy group), preferably a C₁₋₇alkyl group.

Alkoxy: —OR, wherein R is an alkyl group, for example, a C₁₋₇ alkyl group. Examples of C₁₋₇ alkoxy groups include, but are not limited to, —OMe (methoxy), —OEt (ethoxy), —O(nPr) (n-propoxy), —O(iPr) (isopropoxy), —O(nBu) (n-butoxy), —O(sBu) (sec-butoxy), —O(iBu) (isobutoxy), and —O(tBu) (tert-butoxy).

Acetal: —CH(OR)₂, wherein each R is independently an acetal substituents, for example, a C₁₋₇ alkyl group, a C₃₋₂₀ heterocyclyl group, or a C₅₋₂₀ aryl group, preferably a C₁₋₇ alkyl group, or, in the case of a “cyclic” acetal group, R¹ and R², taken together with the two oxygen atoms to which they are attached, and the carbon atoms to which they are attached, form a heterocyclic ring having from 4 to 8 ring atoms. Examples of acetal groups include, but are not limited to, —CH(OMe)₂, —CH(OEt)₂, and —CH(OMe)(OEt).

Hemiacetal: —CH(OH)(OR), wherein R is a hemiacetal substituent, for example, a C₁₋₇ alkyl group, a C₃₋₂₀ heterocyclyl group, or a C₅₋₂₀ aryl group, preferably a C₁₋₇ alkyl group. Examples of hemiacetal groups include, but are not limited to, —CH(OH)(OMe) and —CH(OH)(OEt).

Ketal: —CR(OR)₂, where each R is defined as for acetals, and each R is independently a ketal substituent other than hydrogen, for example, a C₁₋₇ alkyl group, a C₃₋₂₀ heterocyclyl group, or a C₅₋₂₀ aryl group, preferably a C₁₋₇ alkyl group. Examples ketal groups include, but are not limited to, —C(Me)(OMe)₂, —C(Me)(OEt)₂, —C(Me)(OMe)(OEt), —C(Et)(OMe)₂, —C(Et)(OEt)₂, and —C(Et)(OMe)(OEt).

Hemiketal: —CR(OH)(OR), where R is as defined for hemiacetals, and R is a hemiketal substituent other than hydrogen, for example, a C₁₋₇ alkyl group, a C₃₋₂₀ heterocyclyl group, or a C₅₋₂₀ aryl group, preferably a C₁₋₇ alkyl group. Examples of hemiacetal groups include, but are not limited to, —C(Me)(OH)(OMe), —C(Et)(OH)(OMe), —C(Me)(OH)(OEt), and —C(Et)(OH)(OEt).

Oxo (keto, -one): ═O.

Thione (thioketone): ═S.

Imino (imine): ═NR, wherein R is an imino substituent, for example, hydrogen, C₁₋₇ alkyl group, a C₃₋₂₀ heterocyclyl group, or a C₅₋₂₀ aryl group, preferably hydrogen or a C₁₋₇ alkyl group. Examples of ester groups include, but are not limited to, ═NH, ═NMe, ═NEt, and ═NPh.

Formyl (carbaldehyde, carboxaldehyde): —C(═O)H.

Acyl (keto): —C(═O)R, wherein R is an acyl substituent, for example, a C₁₋₇ alkyl group (also referred to as C₁₋₇ alkylacyl or C₁₋₇ alkanoyl), a C₃₋₂₀ heterocyclyl group (also referred to as C₃₋₂₀ heterocyclylacyl), a C₅₋₂₀ aryl group (also referred to as C₅₋₂₀ arylacyl), preferably a C₁₋₇ alkyl group or a halo. Examples of acyl groups include, but are not limited to, —C(═O)CH₃ (acetyl), —C(═O)CH₂CH₃ (propionyl), —C(═O)C(CH₃)₃ (t-butyryl), —C(═O)Ph (benzoyl, phenone), —C(═O)Cl.

Carboxy (carboxylic acid): —C(═O)OH.

Thiocarboxy (thiocarboxylic acid): —C(═S)SH.

Thiolocarboxy (thiolocarboxylic acid): —C(═O)SH.

Thionocarboxy (thionocarboxylic acid): —C(═S)OH.

Imidic acid: —C(═NH)OH.

Hydroxamic acid: —C(═NOH)OH.

Ester (carboxylate, carboxylic acid ester, oxycarbonyl): —C(═O)OR, wherein R is an ester substituent, for example, a C₁₋₇ alkyl group, a C₃₋₂₀ heterocyclyl group, or a C₅₋₂₀ aryl group, preferably a C₁₋₇ alkyl group. Examples of ester groups include, but are not limited to, —C(═O)OCH₃, —C(═O)OCH₂CH₃, —C(═O)OC(CH₃)₃, and —C(═O)OPh.

Acyloxy (reverse ester): —OC(═O)R, wherein R is an acyloxy substituent, for example, a C₁₋₇ alkyl group, a C₃₋₂₀ heterocyclyl group, or a C₅₋₂₀ aryl group, preferably a C₁₋₇ alkyl group. Examples of acyloxy groups include, but are not limited to, —OC(═O)CH₃ (acetoxy), —OC(═O)CH₂CH₃, —OC(═O)C(CH₃)₃, —OC(═O)Ph, and —OC(═O)CH₂Ph.

Oxycarboyloxy: —OC(═O)OR, wherein R is an ester substituent, for example, a C₁₋₇ alkyl group, a C₃₋₂₀ heterocyclyl group, or a C₅₋₂₀ aryl group, preferably a C₁₋₇ alkyl group. Examples of ester groups include, but are not limited to, —OC(═O)OCH₃, —OC(═O)OCH₂CH₃, —OC(═O)OC(CH₃)₃, and —OC(═O)OPh.

Amino: —NR₂, wherein each R is independently an amino substituent, for example, hydrogen, a C₁₋₇ alkyl group (also referred to as C₁₋₇ alkylamino or di-C₁₋₇ alkylamino), a C₃₋₂₀ heterocyclyl group, or a C₅₋₂₀ aryl group, preferably H or a C₁₋₇ alkyl group, or, in the case of a “cyclic” amino group, both R's, taken together with the nitrogen atom to which they are attached, form a heterocyclic ring having from 4 to 8 ring atoms. Amino groups may be primary (—NH₂), secondary (—NHR), or tertiary (—NHR₂), and in cationic form, may be quaternary (—⁺NR₃). Examples of amino groups include, but are not limited to, —NH₂, —NHCH₃, —NHC(CH₃)₂, —N(CH₃)₂, —N(CH₂CH₃)₂, and —NHPh. Examples of cyclic amino groups include, but are not limited to, aziridino, azetidino, pyrrolidino, piperidino, piperazino, morpholino, and thiomorpholino.

Amido (carbamoyl, carbamyl, aminocarbonyl, carboxamide): —C(═O)NR₂, wherein each R is independently an amino substituent, as defined for amino groups. Examples of amido groups include, but are not limited to, —C(═O)NH₂, —C(═O)NHCH₃, —C(═O)N(CH₃)₂, —C(═O)NHCH₂CH₃, and —C(═O)N(CH₂CH₃)₂, as well as amido groups in which both R's, together with the nitrogen atom to which they are attached, form a heterocyclic structure as in, for example, piperidinocarbonyl, morpholinocarbonyl, thiomorpholinocarbonyl, and piperazinocarbonyl.

Thioamido (thiocarbamyl): —C(═S)NR₂, wherein each R is independently an amino substituent, as defined for amino groups. Examples of amido groups include, but are not limited to, —C(═S)NH₂, —C(═S)NHCH₃, —C(═S)N(CH₃)₂, and —C(═S)NHCH₂CH₃.

Acylamido (acylamino): —NR¹⁰C(═O)R¹¹, wherein R¹⁰ is an amide substituent, for example, hydrogen, a C₁₋₇ alkyl group, a C₃₋₂₀ heterocyclyl group, or a C₅₋₂₀ aryl group, preferably hydrogen or a C₁₋₇ alkyl group, and R¹¹ is an acyl substituent, for example, a C₁₋₇ alkyl group, a C₃₋₂₀ heterocyclyl group, or a C₅₋₂₀aryl group, preferably hydrogen or a C₁₋₇ alkyl group. Examples of acylamide groups include, but are not limited to, —NHC(═O)CH₃, —NHC(═O)CH₂CH₃, and —NHC(═O)Ph. R¹⁰ and R¹¹ may together form a cyclic structure, as in, for example, succinimidyl, maleimidyl, and phthalimidyl:

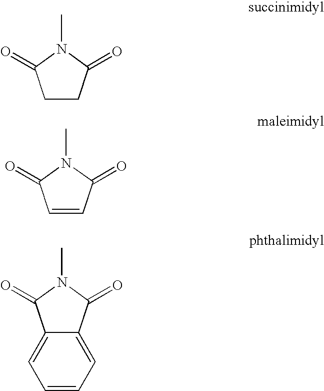

Aminocarbonyloxy: —OC(═O)NR₂, wherein each R is independently an amino substituent, as defined for amino groups. Examples of aminocarbonyloxy groups include, but are not limited to, —OC(═O)NH₂, —OC(═O)NHMe, —OC(═O)NMe₂, and —OC(═O)NEt₂.

Ureido: —N(R¹²)CONR₂ wherein each R is independently an amino substituent, as defined for amino groups, and R¹² is a ureido substituent, for example, hydrogen, a C₁₋₇ alkyl group, a C₃₋₂₀ heterocyclyl group, or a C₅₋₂₀ aryl group, preferably hydrogen or a C₁₋₇ alkyl group. Examples of ureido groups include, but are not limited to, —NHCONH₂, —NHCONHMe, —NHCONHEt, —NHCONMe₂, —NHCONEt₂, —NMeCONH₂, —NMeCONHMe, —NMeCONHEt, —NMeCONMe₂, and —NMeCONEt₂.

Guanidino: —NH—C(═NH)NH₂.

Imino: ═NR, wherein R is an imino substituent, for example, for example, hydrogen, a C₁₋₇ alkyl group, a C₃₋₂₀ heterocyclyl group, or a C₅₋₂₀ aryl group, preferably H or a C₁₋₇alkyl group. Examples of imino groups include, but are not limited to, ═NH, ═NMe, and ═NEt.

Amidine (amidino): —C(═NR)NR₂, wherein each R is independently an amidine substituent, for example, hydrogen, a C₁₋₇ alkyl group, a C₃₋₂₀ heterocyclyl group, or a C₅₋₂₀ aryl group, preferably H or a C₁₋₇ alkyl group. Examples of amidine groups include, but are not limited to, —C(═NH)NH₂, —C(═NH)NMe₂, and —C(═NMe)NMe₂.

Nitro: —NO₂.

Nitroso: —NO.

Azido: —N₃.

Cyano (nitrile, carbonitrile): —CN.

Isocyano: —NC.

Cyanato: —OCN.

Isocyanato: —NCO.

Thiocyano (thiocyanato): —SCN.

Isothiocyano (isothiocyanato): —NCS.

Sulfhydryl (thiol, mercapto): —SH.

Thioether (sulfide): —SR, wherein R is a thioether substituent, for example, a C₁₋₇ alkyl group (also referred to as a C₁₋₇alkylthio group), a C₃₋₂₀ heterocyclyl group, or a C₅₋₂₀ aryl group, preferably a C₁₋₇ alkyl group. Examples of C₁₋₇ alkylthio groups include, but are not limited to, —SCH₃ and —SCH₂CH₃.

Disulfide: —SS—R, wherein R is a disulfide substituent, for example, a C₁₋₇ alkyl group, a C₃₋₂₀ heterocyclyl group, or a C₅₋₂₀ aryl group, preferably a C₁₋₇ alkyl group (also referred to herein as C₁₋₇ alkyl disulfide). Examples of C₁₋₇ alkyl disulfide groups include, but are not limited to, —SSCH₃ and —SSCH₂CH₃.

Sulfine (sulfinyl, sulfoxide): —S(═O)R, wherein R is a sulfine substituent, for example, a C₁₋₇ alkyl group, a C₃₋₂₀ heterocyclyl group, or a C₅₋₂₀ aryl group, preferably a C₁₋₇ alkyl group. Examples of sulfine groups include, but are not limited to, —S(═O)CH₃ and —S(═O)CH₂CH₃.

Sulfone (sulfonyl): —S(═O)₂R, wherein R is a sulfone substituent, for example, a C₁₋₇ alkyl group, a C₃₋₂₀ heterocyclyl group, or a C₅₋₂₀ aryl group, preferably a C₁₋₇ alkyl group, including, for example, a fluorinated or perfluorinated C₁₋₇ alkyl group. Examples of sulfone groups include, but are not limited to, —S(═O)₂CH₃ (methanesulfonyl, mesyl), —S(═O)₂CF₃ (triflyl), —S(═O)₂CH₂CH₃ (esyl), —S(═O)₂C₄F₉ (nonaflyl), —S(═O)₂CH₂CF₃ (tresyl), —S(═O)₂CH₂CH₂NH₂ (tauryl), —S(═O)₂Ph (phenylsulfonyl, besyl), 4-methylphenylsulfonyl (tosyl), 4-chlorophenylsulfonyl (closyl), 4-bromophenylsulfonyl (brosyl), 4-nitrophenyl (nosyl), 2-naphthalenesulfonate (napsyl), and 5-dimethylamino-naphthalen-1-ylsulfonate (dansyl).

Sulfinic acid (sulfino): —S(═O)OH, —SO₂H.

Sulfonic acid (sulfo): —S(═O)₂OH, —SO₃H.

Sulfinate (sulfinic acid ester): —S(═O)OR; wherein R is a sulfinate substituent, for example, a C₁₋₇ alkyl group, a C₃₋₂₀ heterocyclyl group, or a C₅₋₂₀ aryl group, preferably a C₁₋₇ alkyl group. Examples of sulfinate groups include, but are not limited to, —S(═O)OCH₃ (methoxysulfinyl; methyl sulfinate) and —S(═O)OCH₂CH₃ (ethoxysulfinyl; ethyl sulfinate).

Sulfonate (sulfonic acid ester): —S(═O)₂OR, wherein R is a sulfonate substituent, for example, a C₁₋₇ alkyl group, a C₃₋₂₀ heterocyclyl group, or a C₅₋₂₀ aryl group, preferably a C₁₋₇ alkyl group. Examples of sulfonate groups include, but are not limited to, —S(═O)₂OCH₃ (methoxysulfonyl; methyl sulfonate) and —S(═O)₂OCH₂CH₃ (ethoxysulfonyl; ethyl sulfonate).

Sulfinyloxy: —OS(═O)R, wherein R is a sulfinyloxy substituent, for example, a C₁₋₇ alkyl group, a C₃₋₂₀ heterocyclyl group, or a C₅₋₂₀ aryl group, preferably a C₁₋₇ alkyl group. Examples of sulfinyloxy groups include, but are not limited to, —OS(═O)CH₃ and —OS(═O)CH₂CH₃.

Sulfonyloxy: —OS(═O)₂R, wherein R is a sulfonyloxy substituent, for example, a C₁₋₇ alkyl group, a C₃₋₂₀ heterocyclyl group, or a C₅₋₂₀ aryl group, preferably a C₁₋₇ alkyl group. Examples of sulfonyloxy groups include, but are not limited to, —OS(═O)₂CH₃ (mesylate) and —OS(═O)₂CH₂CH₃ (esylate).

Sulfate: —OS(═O)₂OR; wherein R is a sulfate substituent, for example, a C₁₋₇ alkyl group, a C₃₋₂₀ heterocyclyl group, or a C₅₋₂₀ aryl group, preferably a C₁₋₇ alkyl group. Examples of sulfate groups include, but are not limited to, —OS(═O)₂OCH₃ and —SO(═O)₂OCH₂CH₃.

Sulfamyl (sulfamoyl; sulfinic acid amide; sulfinamide): —S(═O)NR₂, wherein each R is independently an amino substituent, as defined for amino groups. Examples of sulfamyl groups include, but are not limited to, —S(═O)NH₂, —S(═O)NH(CH₃), —S(═O)N(CH₃)₂, —S(═O)NH(CH₂CH₃), —S(═O)N(CH₂CH₃)₂, and —S(═O)NHPh.

Sulfonamido (sulfinamoyl; sulfonic acid amide; sulfonamide): —S(═O)₂NR₂, wherein each R is independently an amino substituent, as defined for amino groups. Examples of sulfonamido groups include, but are not limited to, —S(═O)₂NH₂, —S(═O)₂NH(CH₃), —S(═O)₂N(CH₃)₂, —S(═O)₂NH(CH₂CH₃), —S(═O)₂N(CH₂CH₃)₂, and —S(═O)₂NHPh.

Sulfamino: —NRS(═O)₂OH, wherein R is an amino substituent, as defined for amino groups. Examples of sulfamino groups include, but are not limited to, —NHS(═O)₂OH and —N(CH₃)S(═O)₂OH.

Sulfonamino: —NR¹³S(═O)₂R, wherein R¹³ is an amino substituent, as defined for amino groups, and R is a sulfonamino substituent, for example, a C₁₋₇ alkyl group, a C₃₋₂₀ heterocyclyl group, or a C₅₋₂₀ aryl group, preferably a C₁₋₇ alkyl group. Examples of sulfonamino groups include, but are not limited to, —NHS(═O)₂CH₃ and —N(CH₃)S(═O)₂C₆H₅.

Sulfinamino: —NR¹³S(═O)R, wherein R¹³ is an amino substituent, as defined for amino groups, and R is a sulfinamino substituent, for example, a C₁₋₇ alkyl group, a C₃₋₂₀ heterocyclyl group, or a C₅₋₂₀ aryl group, preferably a C₁₋₇ alkyl group. Examples of sulfinamino groups include, but are not limited to, —NHS(═O)CH₃ and —N(CH₃)S(═O)C₆H₅.

Phosphino (phosphine): —PR₂, wherein each R is independently a phosphino substituent, for example, —H, a C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphino groups include, but are not limited to, —PH₂, —P(CH₃)₂, —P(CH₂CH₃)₂, —P(t-Bu)₂, and —P(Ph)₂.

Phospho: —P(═O)₂.

Phosphinyl (phosphine oxide): —P(═O)R₂, wherein each R is independently a phosphinyl substituent, for example, a C₁₋₇ alkyl group, a C₃₋₂₀ heterocyclyl group, or a C₅₋₂₀ aryl group, preferably a C₁₋₇ alkyl group or a C₅₋₂₀ aryl group. Examples of phosphinyl groups include, but are not limited to, —P(═O)(CH₃)₂, —P(═O)(CH₂CH₃)₂, —P(═O)(t-Bu)₂, and —P(═O)(Ph)₂.

Phosphonic acid (phosphono): —P(═O)(OH)₂.

Phosphonate (phosphono ester): —P(═O)(OR)₂, wherein each R is independently a phosphonate substituent, for example, —H, a C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphonate groups include, but are not limited to, —P(═O)(OCH₃)₂, —P(═O)(OCH₂CH₃)₂, —P(═O)(O-t-Bu)₂, and —P(═O)(OPh)₂.

Phosphoric acid (phosphonooxy): —OP(═O)(OH)₂.

Phosphate (phosphonooxy ester): —OP(═O)(OR)₂, wherein each R is independently a phosphate substituent, for example, —H, a C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphate groups include, but are not limited to, —OP(═O)(OCH₃)₂, —OP(═O)(OCH₂CH₃)₂, —OP(═O)(O-t-Bu)₂, and —OP(═O)(OPh)₂.

Phosphorous acid: —OP(OH)₂.

Phosphite: —OP(OR)₂, wherein each R is independently a phosphite substituent, for example, —H, a C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphite groups include, but are not limited to, —OP(OCH₃)₂, —OP(OCH₂CH₃)₂, —OP(O-t-Bu)₂, and —OP(OPh)₂.

Phosphoramidite: —OP(OR)—NR₂, wherein each R is independently a phosphoramidite substituent, for example, —H, a (optionally substituted) C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphoramidite groups include, but are not limited to, —OP(OCH₂CH₃)—N(CH₃)₂, —OP(OCH₂CH₃)—N(i-Pr)₂, and —OP(OCH₂CH₂CN)—N(i-Pr)₂.

Phosphoramidate: —OP(═O)(OR)—NR₂, wherein each R is independently a phosphoramidate substituent, for example, —H, a (optionally substituted) C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphoramidate groups include, but are not limited to, —OP(═O)(OCH₂CH₃)—N(CH₃)₂, —OP(═O)(OCH₂CH₃)—N(i-Pr)₂, and —OP(═O)(OCH₂CH₂CN)—N(i-Pr)₂.

Alkylene

The term “alkylene”, as used herein, pertains to a bidentate moiety obtained by removing two hydrogen atoms, either both from the same carbon atom, or one from each of two different carbon atoms, of a hydrocarbon compound having from 1 to 12 carbon atoms (unless otherwise specified), which may be aliphatic or alicyclic, and which may be saturated, partially unsaturated, or fully unsaturated. Thus, the term “alkylene” includes the sub-classes alkenylene, alkynylene, cycloalkylene, etc., discussed below.

Examples of linear saturated alkylene groups include, but are not limited to, —(CH₂)ₙ— where n is an integer from 3 to 12, for example, —CH₂CH₂CH₂— (propylene), —CH₂CH₂CH₂CH₂— (butylene), —CH₂CH₂CH₂CH₂CH₂— (pentylene) and —CH₂CH₂CH₂CH—₂CH₂CH₂CH₂— (heptylene).

Examples of branched saturated alkylene groups include, but are not limited to, —CH(CH₃)CH₂—, —CH(CH₃)CH₂CH₂—, —CH(CH₃)CH₂CH₂CH₂—, —CH₂CH(CH₃)CH₂—, —CH₂CH(CH₃)CH₂CH₂—, —CH(CH₂CH₃)—, —CH(CH₂CH₃)CH₂—, and —CH₂CH(CH₂CH₃)CH₂—.

Examples of linear partially unsaturated alkylene groups (alkenylene, and alkynylene groups) include, but are not limited to, —CH═CH—CH₂—, —CH₂—CH═CH₂—, —CH═CH—CH₂—CH₂—, —CH═CH—CH₂—CH₂—CH₂—, —CH═CH—CH═CH—, —CH═CH—CH═CH—CH₂—, —CH═CH—CH═CH—CH₂—CH₂—, —CH═CH—CH₂—CH═CH—, —CH═CH—CH₂—CH₂—CH═CH—, and —CH₂—C≡C—CH₂—.

Examples of branched partially unsaturated alkylene groups (alkenylene and alkynylene groups) include, but are not limited to, —C(CH₃)═CH—, —C(CH₃)═CH—CH₂—, —CH═CH—CH(CH₃)— and —C≡C—CH(CH₃)—.

Examples of alicyclic saturated alkylene groups (cycloalkylenes) include, but are not limited to, cyclopentylene (e.g. cyclopent-1,3-ylene), and cyclohexylene (e.g. cyclohex-1,4-ylene).

Examples of alicyclic partially unsaturated alkylene groups (cycloalkylenes) include, but are not limited to, cyclopentenylene (e.g. 4-cyclopenten-1,3-ylene), cyclohexenylene (e.g. 2-cyclohexen-1,4-ylene; 3-cyclohexen-1,2-ylene; 2,5-cyclohexadien-1,4-ylene).

The term “C₁₋₃ alkylene” as used herein, is an alkylene as defined above group and having from 1 to 3 carbon atoms.

The term “GacS/GacA-type system” refers to the signal transduction system in D. dadantii as well as homologous systems in other bacteria (e.g. E. coli (BarA/UvrY), Pectobacterium spp., S. typhimurium (BarA/SirA), Pseudomonas spp. (GacS/GacA), and Legionella pneumophila (LetS/LetA), Vibrio species) that have a similar structure and function to the GacS/GacA system of D. dadantii, even though the homologous regulatory system may be known by a different name in other bacteria. Similarly, “GacA-type polypeptide” and “GacS-type polypeptide” refer to the respective polypeptides in D. dadantii as well as homologous polypeptides having similar structure and function in other bacteria.

The term “HrpX/HrpY-type system” refers to the signal transduction system in D. dadantii as well as homologous systems in other bacteria that have a similar structure and function to the HrpX/HrpY system of D. dadantii, even though the homologous regulatory system may be known by a different name in other bacteria. Similarly, “HrpY-type polypeptide” and “HrpX-type polypeptide” refer to the respective polypeptides in D. dadantii as well as homologous polypeptides having similar structure and function in other bacteria.

The term “Rsm-type system” refers to the regulator of secondary metabolism (Rsm) system of D. dadantii as well as homologous systems in other bacteria that have a similar structure and function to the Rsm system of D. dadantii, even though the homologous regulatory system may be known by a different name in other bacteria.

The term “T3SS-type system” refers to the type III secretion system (T3SS) of D. dadantii as well as homologous systems in other bacteria that have a similar structure and function to the T3SS of D. dadantii, even though the homologous regulatory system may be known by a different name in other bacteria.

As used herein, the terms “reducing” or “reduced” are used relative to an untreated sample or other suitable control. For example, “reducing expression” of a polynucleotide in a bacterium in response to contacting the bacterium with a test compound means lowering the amount of expression of the polynucleotide (e.g. lowering the amount of polynucleotide-encoded mRNA or protein formed) relative to the level of expression of the polynucleotide in the same bacterium under control conditions. The control conditions may include exposing the bacterium to the same conditions without contacting the bacterium with the test compound.

“Reducing virulence” in a bacterium refers to altering expression of genes associated with virulence, including regulators of virulence. Reducing virulence also refers to physical and biochemical manifestations of virulence including those manifestations associated with any step of the bacterial life cycle when it is associated with a host, including without limitation the adherence, invasion, replication, evasion of host defenses, and transmittal to a new host. Reduced bacterial virulence may be manifested in the form of reduced symptoms in a host, and thus may be detected by monitoring the host for a reduced reaction to the bacteria associated therewith. Reduced virulence may arise as a result of either inhibition or stimulation of a two-component regulatory system such as a GacS/GacA-type system or a HrpX/HrpY-type system, which could lead to increases or decreases of polynucleotide and polypeptide production. For example, reduced virulence may be associated with increase production of a repressor, reduced production of a transcription factor, or increased production of enzymes or toxins. Regulation of bacterial virulence may lead to alterations in the production of pectinase, exoprotease, syringomycin, syringolin, alginate, tolaasin, siderophores, pyocyanin, cyanide, lipase, type III secretion system (T3SS) genes, cholera toxin, polyhydroxybutyrate, or a polynucleotide controlled by a GacS/GacA-type system or a HrpX/HrpY-type system in a Gram negative bacterium. A reduction in virulence may be at least about a 1% reduction, at least about a 10% reduction, at least about a 20% reduction, at least about a 30% reduction, at least about a 40% reduction, at least about a 50% reduction, at least about a 60% reduction, at least about a 70% reduction, at least about a 80% reduction, at least about a 90% reduction, or at least about a 100% reduction of virulence, as measured by any assay described herein or known to those of skill in the art, when measured against a suitable control.

“Components” of a GacS/GacA-type system, a HrpX/HrpY-type system, a T3SS-type system, and a Rsm-type system include without limitation polynucleotides and polypeptides that are part of the respective systems (including genes and gene products of the named operons) as well as polynucleotides, polypeptides, and other molecules that regulate the systems including genes and gene products that are upstream or downstream of the system. “Components” also includes molecules that are products of the genes or gene products of the systems as well as genes or gene products that generate posttranslational modifications of polynucleotides or polypeptides of the systems. A “regulator” is a component that changes (increases or decreases) an expression or activity level of a component. A “repressor” is a component that decreases an expression or activity level of a component, arising from either an increase or a decrease in the amount or activity level of the repressor. An “effector” is a component that puts into effect the activity of the system, e.g. exoenzymes of the T3SS-type system are nonlimiting examples of effectors. A component is “associated with virulence” if a change in an amount or activity of the component leads, directly or indirectly, to an increase or reduction in some aspect of bacterial virulence.

A “phenylpropanoid-type inhibitory compound” as used herein includes phenylpropanoid compounds, such as p-coumaric acid or cinnamyl alcohol, that reduce bacterial virulence. In addition, “phenylpropanoid-type inhibitory compound” also includes phenylpropanoid derivative that have been found to be “active compounds,” i.e. compounds that have shown through screening to have bacterial virulence-reducing activity.

A bacterium is “associated with” (or “associated therewith”) a host or subject such as a plant or animal (including a human) when the bacterium is in or on the host or subject. For a plant host or subject, an associated bacterium can be on a plant part such as a root, stem, leaf, flower, or fruit of the plant, or in the soil adjacent to the roots or base of the stem. For an animal host or subject, an associated bacterium can be on the outer surface of the animal or on an inner surface such as an intestinal surface, or the associated bacterium can be within the animal, e.g. in a tissue or fluid of the animal or any other internal portion of the animal.

In one embodiment, a phenylpropanoid derivative may be a compound of formula (II):

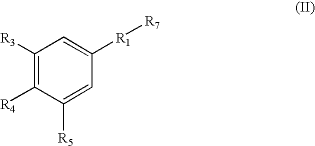

wherein R₁ is an alkylene;

R₃, R₄, and R₅ are independently selected from hydrogen, alkyl, alkenyl, alkynyl, cycloalkyl, heterocyclyl, aryl, heteroaryl, halo, hydroxy, ether, alkoxy, acetal, hemiacetal, ketal, hemiketal, formyl, acyl, carboxy, thiocarboxy, thiolcarboxy, thionocarboxy, imidic acid, hydroxamic acid, ester, acyloxy, oxycarboyloxy, amino, amido, thioamido, acylamido, aminocarbonyloxy, ureido, guanidine, amindino, nitro, nitroso, azido, cyano, isocyano, isocyanato, thiocyano, isothiocyano, sulfhydryl, thioether, disulfide, sulfine, sulfonyl, sulfinic acid, sulfonic acid, sulfinate, sulfonate, sulfinyloxy, sulfonyloxy, sulfate, sulfamyl, sulfonamide, sulfamino, sulfonamino, sulfinamino, phosphino, phosphor, phosphinyl, phosphono, phosphonate, phosphonooxy, phosphate, phosphorous acid, phosphite, phosphoramidite, or phosphoramidate, wherein two of R₃, R₄, and R₅ optionally are linked together to form a ring;

and

R₇ is hydroxy, acetal, hemiacetal, ketal, hemiketal, formyl, acyl, carboxy, thiocarboxy, thiolcarboxy, thionocarboxy, imidic acid, hydroxamic acid, ester, acyloxy, oxycarboyloxy, amino, amido, thioamido, acylamido, aminocarbonyloxy, ureido, guanidine, amindino, nitro, nitroso, azido, cyano, isocyano, isocyanato, thiocyano, isothiocyano, sulfhydryl, thioether, disulfide, sulfine, sulfonyl, sulfinic acid, sulfonic acid, sulfinate, sulfonate, sulfinyloxy, sulfonyloxy, sulfate, sulfamyl, sulfonamide, sulfamino, sulfonamino, sulfinamino, phosphino, phosphor, phosphinyl, phosphono, phosphonate, phosphonooxy, phosphate, phosphorous acid, phosphite, phosphoramidite, or phosphoramidate.

Suitably, R₃ and R₅ are hydrogen, R₄ is hydrogen, hydroxy, sulfhydryl or halo, and R₇ is hydroxy, carboxy or formyl.

The assays may be carried out on intact bacteria or on isolated bacterial components. For example, receptor portions of the GacS/GacA-type system or HrpX/HrpY-type system may be reconstituted in vitro, e.g. in membrane microsomes, or as isolated, soluble protein components. In one embodiment, the assay includes measurement of binding affinity of a test compound to the GacS-type or HrpX-type polypeptide.

The assays may also be conducted on genetically-engineered bacteria in which the GacS/GacA-type system or HrpX/HrpY-type system is coupled to a reporter. Thus, a test compound can be screened by contacting the compound with an appropriately engineered bacterium so that the binding and/or activation of the GacS/GacA pathway by the compound will be directly reported without the need to assay a downstream target or effector such as a component of the Rsm pathway or T3SS system. The reporter in the genetically-engineered bacterium could be linked to any number of known fluorescent or colorimetric assays, e.g. green fluorescent protein (GFP), to make possible rapid screening of large numbers of compounds.

The bacterium can be any one of a number of virulent bacterial species or strains, including those bacterial species or strains having at least one of a GacS/GacA-type system, a HrpX/HrpY-type system, a Rsm-type system, and/or a T3SS-type system. The suitable bacterial species or strains include without limitation Pseudomonas spp., Erwinia-related strains, Azotobacter vinelandii, Vibrio cholarae, Salmonella enterica, and Escherichia coli strains. The bacterium may be a Pseudomonas spp including P. aureofaciens, P. chlororaphis, P. fluorescens, P. marginalis, Pseudomonas syringae, P. tolaasii, P. viridiflava, and P. aeruginosa. The bacterium may be an Erwinia-related strain including Dickeya dadantii, Erwinia carotovora, Erwinia atroseptica, and Erwinia amylovora. Dickeya dadantii is a member of the Enterobacteriaceae family, which includes the plant pathogens Pectobacterium carotovora and Erwinia amylovora as well as animal and human pathogens such as E. coli, Salmonella spp., and Yersinia spp.

After phenylpropanoid derivatives have been screened for their efficacy in reducing induction of virulence, those analogs that show effective reduction (also called “active compounds”) will be tested for use in reducing virulence in bacteria that are associated with a subject such as a plant or an animal, including a human. The bacteria may be on the surface of the subject or within the subject or otherwise associated with the subject. Active compounds may be used in a method to treat a subject having a bacterial infection comprising administering to the subject an effective amount of a composition comprising the compound (see Examples). Active compounds may also be applied to a surface to reduce virulence of bacteria associated with the surface.

“Treating” or “treatment,” as used herein in the context of treating a condition, pertains generally to treatment and therapy, whether of a human, an animal (e.g. in veterinary applications), or plants, in which some desired therapeutic effect is achieved, for example, the reduction of the progress of the condition, and includes a reduction in the rate of progress, a halt in the rate of progress, amelioration of the condition, and cure of the condition. Treatment as a prophylactic measure (i.e. prophylaxis) is also included. “Treating” and “treatment” also refer to reducing the symptoms associated with the condition that is being treated.

In addition to the methods listed below, additional methods known to those skilled in the art may be employed as needed in screening and assaying the synthesized compounds. For example, to detect and measure amounts of polypeptides, methods such as SDS-PAGE gel eletrophoresis, Western blotting, and enzyme-linked immunoassays (ELISA) may be used, among other techniques. To detect and measure polynucleotides, polymerase-chain reaction (PCR) as well as eletrophoretic mobility-based methods such as Southern and Northern blotting may be used, among other techniques.

The following non-limiting Examples are intended to be purely illustrative, and show specific experiments that were carried out in accordance with embodiments of the invention:

### EXAMPLES

### Examples 1-3

The Following Methods Apply to Examples 1-3:

Bacterial Strains, Plasmids, Media and Chemicals

The bacterial strains and plasmids used in this study are listed in Table 1. E. coli was grown in LB broth at 37° C. and D. dadantii was grown in minimal hrp-inducing medium (MM) at 28° C. Antibiotics (μg/ml) used were: ampicillin, 100; chloramphenicol, 50; kanamycin, 50; spectinomycin, 50. Primers used for Polymerase Chain Reaction (PCR) in this report are also listed in Table 1. Chinese cabbage purchased in a grocery store and African violet, without visible symptom from pathogen infection were used in this study.

TABLE 1
Strains, plasmids, and DNA primers used in this study.

| Strains, plasmids | | |
|---|---|---|
| and primers | Characters or sequences (5′ to 3′)^{a} | Reference or source |
| Strains | | |
| E. coli | | |
| E. coli DH5α | F φ80lacZΔM15 ΔlacZYA-argF) U169 deoR recAl endAl | Invitrogen, CA |
| | hsdR17 phoA supE44 thi-1 gyrA96 relAl λ | |
| E. coli TOP10 | F mcrA Δmrr-hsdRMS-mcrBC) φ80lacZΔM15 ΔlacX74 | Invitrogen, CA |
| | deoR recAl araD139 Δ(ara-leu)7679 galU galK rpsL | |
| | endA1 nupG | |
| D. dadantii | | |
| Ech3937 | wild type, Saintpaulia (African violet) isolate | Hugouvieux-Cotte- |
| Ech-Rif | Ech3937 rifampicin resistant random mutant | Pattat, N. |
| Ech131 | ΔhrpL:: kan; Km^{R} | Yang et al., 2008 |
| Ech137 | ΔgacA:: kan; Km^{R} | Yang et al., 2004 |
| Ech138 | ΔiaaM:: kan; Km^{R} | Yang et al., 2008 |
| WPP96 | ΔhrpL_{(Δ1-185aa)}:: aadA; Sp^{R}/Sm^{R} | Yang et al., 2007 |
| WPP90 | hrpS:: cat; Cm^{R} | Yap et al., 2005 |
| WPP67 | hrpX:: aadA; Sp^{R}/Sm^{R} | Yap et al., 2005 |
| WPP92 | hrpY:: kan; Km^{R} | Yap et al., 2005 |
| Ech3937 (pAT) | Ech3937 containing pPROBE-AT | Yap et al., 2005 |
| Ech3937 | Ech3937 containing pdspE; Ap^{R} | Peng et al., 2006 |
| (pdspE) | ΔhrpL:: kan containing pdspE; Ap^{R} Km^{R} | Yang et al., 2004 |
| Ech131 (pdspE) | Ech3937 containing phrpA; Ap^{R} | Peng et al., 2006 |
| Ech3937 | WPP96 containing phrpA; Ap^{R} Sp^{R} | This work |
| (phrpA) | Ech3937 containing phrpN; Ap^{R} | This work |
| WPP96 (phrpA) | WPP96 containing phrpN; Ap^{R} Sp^{R} | Yang et al., 2007 |
| Ech3937 | Ech3937 containing phrpL; Ap^{R} | Yang et al., 2007 |
| (phrpN) | WPP96 containing phrpL; Ap^{R} Sp^{R} | Yang et al., 2007 |
| WPP96 (phrpN) | Ech3937 containing phrpS; Ap^{R} | Yang et al., 2007 |
| Ech3937 (phrpL) | WPP96 containing phrpS; Ap^{R} Sp^{R} | This work |
| WPP96 (phrpL) | Ech3937 containing pmrp; Ap^{R} | This work |
| Ech3937 (phrpS) | Ech-Rif containing phrpA; Ap^{R} | Peng et al., 2006 |
| WPP96 (phrpS) | Ech137 containing phrpA; Ap^{R} Km^{R} | Yang et al., 2008 |
| Rch3937 (pmrp) | Ech3937 containing phrpN; Ap^{R} | Yang et al., 2008 |
| Ech-Rif (phrpA) | Ech138 containing phrpN; Ap^{R} Km^{R} | Yang et al., 2007 |
| Ech137 (phrpA) | WPP90 containing phrpN; Ap^{R} Sp^{R} | Yang et al., 2007 |
| Ech3937 | WPP67 containing phrpN; Ap^{R} Sp^{R} | This work |
| (phrpN) | WPP92 containing phrpN; Ap^{R} Sp^{R} | This work |
| Ech138 (phrpN) | | This work |
| WPP90 (phrpN) | | |
| WPP67 (phrpN) | | |
| WPP92 (phrpN) | | |
| Plasmids | | |
| pPROBE-AT | Promoter-probe vector, Ap^{R} | Miller et al., 1997, 2000 |
| pCR2.1-TOPO | PCR cloning vector, Ap^{R} Km^{R} | Invitrogen, CA |
| pdspE | pProbe-AT derivative with PCR fragment containing dspE | Yang et al., 2004 |
| | promoter region, Ap^{R} | |
| phrpA | pProbe-AT derivative with PCR fragment containing 412- | This work |
| | bp hrpA promoter region, Ap^{R} | |
| phrpN | pProbe-AT derivative with PCR fragment containing hrpN | Yang et al., 2007 |
| | promoter region, Ap^{R} | |
| phrpL | pProbe-AT derivative with PCR fragment containing hrpL | Yang et al., 2007 |
| | promoter region, Ap^{R} | |
| phrpS | pProbe-AT derivative with PCR fragment containing 709- | This work |
| | bp hrpS promoter region, Ap^{R} | |
| Primers | | |
| phrpA_F | GTGCCGATAGCCAGTGAT | This work |
| | (SEQ ID NO: 1) | |
| phrpA_R | TGCTGCTGCGTTAGAAAG | This work |
| | (SEQ ID NO: 2) | |
| phrpS_F | CAGATTGTATTTGCGGATTG | This work |
| | (SEQ ID NO: 3) | |
| phrpS_R | CGGATTCATTGCTATTCCTTAT | This work |
| | (SEQ ID NO: 4) | |
| rplU_RTF | GCGGCAAAATCAAGGCTGAAGTCG | Yang et al., 2007 |
| | (SEQ ID NO: 5) | |
| rplU_RTR | CGGTGGCCAGCCTGCTTACGGTAG | Yang et al., 2007 |
| | (SEQ ID NO: 6) | |
| hrpY_RTF | CGGCGACGGGCGTAATGAA | This work |
| | (SEQ ID NO: 7) | |
| hrpY_RTR | TTTCGGCGATGGCATTGACC | This work |
| | (SEQ ID NO: 8) | |
| hrpS_RTF | TGGAAGGCGAAACCGGCACC | This work |
| | (SEQ ID NO: 9) | |
| hrpS_RTR | GCACGGCGGCGCAGTTCAC | This work |
| | (SEQ ID NO: 10) | |
| hrpL_RTF | GATGATGCTGCTGGATGCCGATGT | Yang et al., 2007 |
| | (SEQ ID NO: 11) | |
| hrpL_RTR | TGCATCAACAGCCTGGCGGAGATA | Yang et al., 2007 |
| | (SEQ ID NO: 12) | |
| hrpA_RTF | CAGCAATGGCAGGCATGCAG | Yang et al., 2007 |
| | (SEQ ID NO: 13) | |
| hrpA_RTR | CTGGCCGTCGGTGATTGAGC | Yang et al., 2007 |
| | (SEQ ID NO: 14) | |
| dspE_RTF | GATGGCGGAGCTGAAATCGTTC | Yang et al., 2007 |
| | (SEQ ID NO: 15) | |
| dspE_RTR | CCTTGCCGGACCGCTTATCATT | Yang et al., 2007 |
| | (SEQ ID NO: 16) | |
| rsmB_RTF | AGAGGGATCGCCAGCAAGGATTGT | This work |
| | (SEQ ID NO: 17) | |
| rsmB RTR | CGTTTGCAGCAGTCCCGCTACC | This work |
| | (SEQ ID NO: 18) | |

^{a}Ap^{R}, ampicillin resistance; Cm^{R}, chloramphenicol resistance; Km^{R}, kanamycin resistance; Sp^{R}, spectinomycin resistance.
Hugouvieux-Cotte-Pattat, N., Condemine, G., Nasser, W., and Reverchon, S. (1996) Regulation of pectinolysis in Erwinia chrysanthemi. Annu Rev Microbiol 50: 213-257.
Miller, W. G., Leveau, J. H. J., and Lindow, S. E. (2000) Improved gfp and inaZ broad-host-range promoter-probe vectors. Mol Plant-Microbe Interact 13: 1243-1250.
Miller, W. G., and Lindow, S. E. (1997) An improved GFP cloning cassette designed for prokaryotic transcriptional fusions. Gene 191: 149-153.
Peng, Q., Yang, S., Charkowski, A. O., Yap, M. N., Steeber, D. A., Keen, N. T., and Yang, C. H. (2006) Population behavior analysis of dspE and pelD regulation in Erwinia chrysanthemi 3937. Mol Plant-Microbe Interact 19: 451-457.
Yang, S., Q. Peng, Q. Zhang, X. Yi, C. J. Choi, R. M. Reedy, A. O. Charkowski, and C. -H. Yang. 2008. Dynamic regulation of GacA in type III secretion system, pectinase gene expression, pellicle formation, and pathogenicity of Dickeya dadantii. Mol. Plant-Microbe Interact. 21: 133-142.
Yang, S., Zhang, Q., Guo, J., Charkowski, A. O., Glick, B. R., Ibekwe, A. M. et al. (2007) Global effect of Indole-3-acetic acid (IAA) biosynthesis on multiple virulence factors of Erwinia chrysanthemi 3937. Appl Environ Microbiol 73: 1079-1088.
Yang, S., Perna, N. T., Cooksey, D. A., Okinaka, Y., Lindow, S. E., Ibekwe, A. M. et al. (2004) Genome-wide identification of plant-upregulated genes of Erwinia chrysanthemi 3937 using a GFP-based IVET leaf array. Mol Plant-Microbe Interact 17: 999-1008.
Yap, M. N., Yang, C. H., Barak, J. D., Jahn, C. E., and Charkowski, A. O. (2005) The Erwinia chrysanthemi type III secretion system is required for multicellular behavior. J Bacteriol 187: 639-648.

FACS Analysis

FACS analysis of promoter activity of dspE, hrpA, hrpL, hrpN, and hrpS was carried out as described (Peng et al., 2006). Briefly, the wild-type Ech3937 and the mutant strains carrying the promoter reporter plasmid were grown on LB broth at 28° C. overnight and transferred to appropriate media. For FACS analysis, samples were collected by centrifugation, washed with 1× phosphate buffer saline (PBS) at 13,000 rpm for 1 min, and re-suspended in 1×PBS to ca 10⁶ CFU/ml prior to being run in a FACS Calibur flow cytometer (BD Biosciences, CA). Three replicates were performed for each treatment.

qRT-PCR Analysis

Bacterial strains were grown in MM. Total RNA from the bacterial cells was isolated by using the TRI reagent method (Sigma, Mo.) and treated with Turbo DNA-free DNase kits (Ambion, TX) as described (Peng et al., 2006). An iScript cDNA Synthesis Kit (Bio-Rad, CA) was used to synthesize cDNA from 0.5 μg of treated total RNA. The Real Master Mix (Eppendorf, Westbury, N.Y.) was used for qRT-PCR reaction to quantify the cDNA level of target genes in different samples. The rplU was used as the endogenous control for data analysis. qRT-PCR data were analyzed using Relative Expression Software Tool as described (Pfaffl, M. W., Horgan, G. W., and Dempfle, L. (2002) Relative expression software tool (REST) for group-wise comparison and statistical analysis of relative expression results in real-time PCR. Nucleic Acids Res 30:e36.).

### Example 1

### T3SS Gene Expression Induced by Plant Phenolic Compounds

Screening of plant up-regulated genes in Ech3937 has demonstrated that dspE and hrpA were expressed in planta. In this study, the expression of dspE of Ech3937 was further compared in bacterial cells grown in a nutrient rich LB medium, a nutrient-limited medium (MM), and the MM supplemented with Chinese cabbage juice (10% V/V7). For this purpose, a GFP reporter plasmid pdspE (pProbe-AT derivative with PCR fragment containing dspE promoter region) was used and cells cultured under different conditions were compared by flow cytometry. A higher total GFP intensity was observed in Ech3937 (pdspE) (Ech3937 cells carrying plasmid pdspE) grown in MM compared with the bacterial cells grown in LB (Table 2). The expression of dspE was further induced in MM supplemented with Chinese cabbage juice in comparison with the bacterial cells grown in MM alone. In addition, low promoter activities of dspE were observed in hrpL mutant Ech131 carrying pdspE grown in MM and MM supplemented with Chinese cabbage (Table 2), suggesting that HrpL is essential for the expression of dspE under inducing conditions.

TABLE 2
The expression of dspE of Dickeya dadantii 3937 (Ech3937)
and the hrpL mutant Ech131 grown in LB, MM, and MM with
Chinese cabbage juice (10% V/V) (MMJ).

| Gene Promoter^{a} | LB | MM | MMJ |
|---|---|---|---|
| Ech3937 (pdspE)^{b} | 3.2 ± 0.2 | 45 ± 1 | 161 ± 4 |
| Ech131 (pdspE) | 2.1 ± 0.2 | 2.1 ± 0.7 | 1.3 ± 0 |
| Ech3937 (pProbe-AT) | 1.9 ± 0.2 | 1.4 ± 0 | 1.2 ± 0 |

^{a}The dspE promoter activities in the wild-type Ech3937 and hrpL mutant Ech131 were compared after 12 h of culture in LB, MM, and MM supplemented with plant juice. GFP intensity was determined on gated populations of bacterial cells by flow cytometry. The fluorescence intensities were an average GFP fluorescence intensity of total bacterial cells.
^{b}Values (Mean Fluorescence Intensity) are representative of two experiments. Three replicates were used in this experiment. The values are the average with the standard deviation.

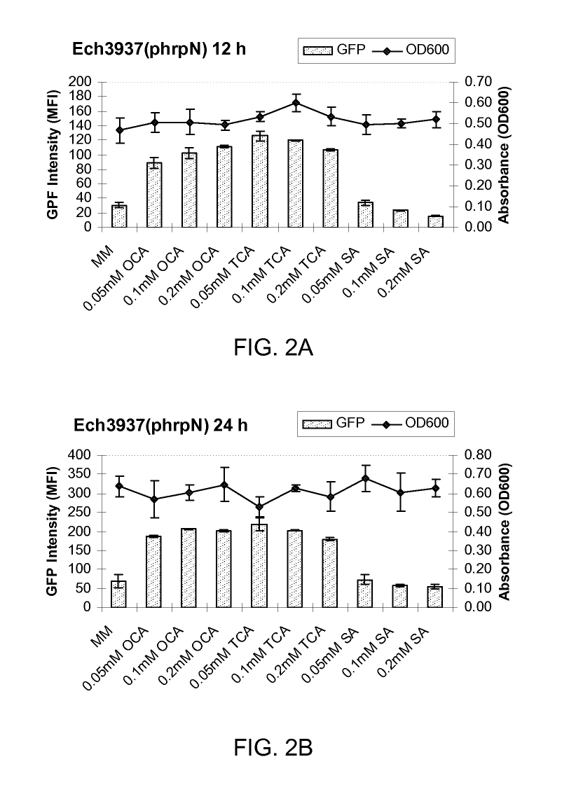

Plant juice induced the expression of T3SS genes of Ech3937, suggesting the existence of compounds in plant tissues that activate the T3SS regulon. Phenolic compounds constitute an important class of organic substances produced by plants. The phenolic compound SA is a signaling molecule that plays a role in host defenses. OCA and TCA are the biosynthetic precursors of SA and are also reported to induce the expression of defense-related genes in plants. OCA, TCA, and SA were examined to elucidate their effects on the expression of T3SS genes. The expression of the T3SS gene hrpN was examined in MM and MM supplemented with OCA, TCA, and SA, at concentrations of 0.05, 0.1, and 0.2 mM, respectively. FIGS. 2A and 2B show the promoter activities of hrpN in Dickeya dadantii 3937 (Ech3937) grown in MM and MM supplemented with 0.05, 0.1, and 0.2 mM OCA, TCA, and SA at 12 h (FIG. 2A) and 24 h (FIG. 2B) post-inoculation. GFP intensity was determined on gated populations of bacterial cells by flow cytometry and analyzed with the Cell Quest software (BD Biosciences, San Jose, Calif.). The growth of Ech3937 in MM supplemented with different concentrations of OCA, TCA and SA was recorded. Compared with MM alone, the average GFP fluorescence intensity of bacterial cells of Ech3937 (phrpN) was increased approximately 4-fold when 0.05 mM of OCA and TCA were added to the medium (FIGS. 2A, 2B). The addition of SA did not result in increased GFP fluorescence intensity of Ech3937 (FIGS. 2A, 2B). No reduction of bacterial growth was observed when OCA, TCA, and SA were added into the MM (FIGS. 2A, 2B).

The concentration of the phenolic compound t-cinnamic acid (TCA) in healthy potato leaves is approximately 0.5 μM and levels in the leaves can rise to approximately 10 uM after exposure to a cell-free culture filtrate (CF) of E. c. carotovora. To investigate whether the level of the phenolic compounds in plants is able to induce the expression of T3SS gene, the expression of hrpN was examined using concentrations of TCA comparable to levels found in plants. Ech3937 (phrpN) was grown in MM supplemented with 0.2, 0.5, 5, and 10 μM of TCA, respectively. Compared with Ech3937 (phrpN) in MM alone, a 1.5- to 1.8-fold increase of GFP intensity was observed in the bacterial cells grown in MM supplemented with 0.2 and 0.5 μM TCA (Table 3). Compared with Ech3937 (phrpN) in MM, a 3- to 3.5-fold higher GFP intensity was observed in the bacterial cells grown in MM supplemented with 5 and 10 μM of TCA (Table 2).

TABLE 3
The expression of hrpN of Dickeya dadantii
3937 (Ech3937) in MM and MM supplemented with
different amount of TCA and SA respectively.

GFP Intensity of Ech3937 (phrpN)^{b}

| | 12 h | 24 h |
|---|---|---|
| MM^{a} | 41.6 ± 3.6 | 91.3 ± 11 |
| TCA^{a} | | |
| 0.2 μM | 65.1 ± 6.8 | 163 ± 25 |
| 0.5 μM | 73.4 ± 4.2 | 158 ± 20 |
| 5 μM | 134 ± 5.6 | 266 ± 14 |
| 10 μM | 147 ± 18 | 284 ± 12 |
| SA^{a} | | |
| 0.2 μM | 53.7 ± 4.0 | 105 ± 28 |
| 0.5 μM | 49.5 ± 5.9 | 99.5 ± 18 |
| 5 μM | 49.9 ± 2.9 | 97.7 ± 9.0 |
| 10 μM | 52.0 ± 2.3 | 104 ± 3.1 |

^{a}Minimal medium (MM) alone and MM supplemented with different concentrations of t-cinnamic acid (TCA) or salicylic acid (SA).
^{b}The promoter activities of hrpN were measured at 12 and 24 h of growing in the media. GFP intensity was determined on gated populations of bacterial cells by flow cytometry. The fluorescence intensities were an average GFP fluorescence intensity of total bacterial cells. Three replicates were used in this experiment. The value (Mean Fluorescence Intensity) is present as the average of three replicates with standard deviation.

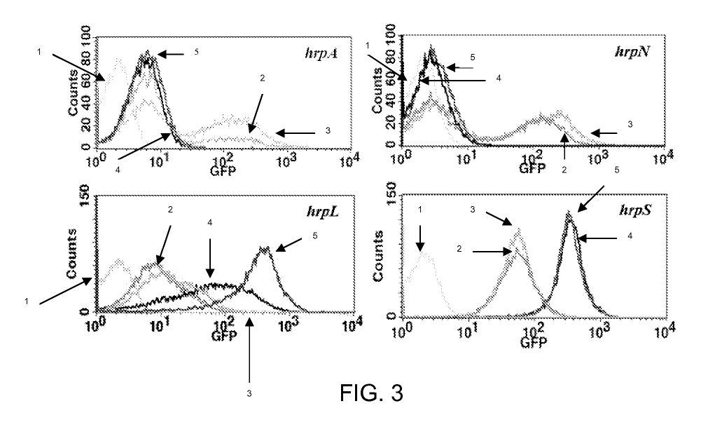

Since OCA and TCA induced the expression of hrpN, the effect of these two phenolic compounds on the expression of hrpA, hrpL, and hrpS was investigated further. FIG. 3 shows the promoter activities of hrpA, hrpN, hrpL, and hrpS in Dickeya dadantii 3937 (Ech3937) and hrpL mutant WPP96 grown in MM and MM supplemented with 0.1 mM OCA 12 h post-grown. GFP intensity was determined on gated populations of bacterial cells by flow cytometry and analyzed with the Cell Quest software (BD Biosciences, San Jose, Calif.). The lines labeled “1” stand for the GFP expression control base level of the Ech3937 containing pPROBE-AT vector; lines labeled “2” stand for the promoter activity of hrpS, hrpL, hrpA and hrpN in Ech3937 in MM; lines labeled “3” stand for the promoter activity of Ech3937 in MM supplemented with 0.1 mM OCA; lines labeled “4” stand for the promoter activity of hrpL mutant WPP96 in MM; lines labeled “5” stand for the promoter activity of hrpL mutant WPP96 in MM supplemented with 0.1 mM OCA. Values are representative of at least two experiments. Three biological replicates were used in this experiment, which had similar results, and one replicate was used for the overlay as displayed. Compared with MM alone, the average GFP fluorescence intensity of bacterial cells of Ech3937 (phrpA) was doubled when 0.1 mM of OCA and TCA were added to the medium (Table 4; FIG. 3). Compared with MM alone, a slightly higher promoter activity of hrpL was observed in Ech3937 (hrpL) grown in MM supplemented with OCA and TCA, respectively. Compared with MM alone, a slightly lower GFP intensity of Ech3937 (phrpS) was observed when the bacterial cells were grown in MM supplemented with OCA and TCA (Table 4; FIG. 3). The mrp, whose protein product has an ATPase conserved domain (2e-06), was used as a reference gene in this study. A slightly higher mrp expression was observed in Ech3937 (pmrp) when the bacterial cells were grown in MM and MM supplemented with 0.1 mM OCA and TCA, respectively (Table 4).

TABLE 4
The expression of hrpA, hrpN, hrpL, hrpS, and mrp
of Dickeya dadantii 3937 (Ech3937) and hrpL mutant
WPP96 in MM, MM supplemented with 0.1 mM OCA (MMOCA),
and MM supplemented with 0.1 mM TCA (MMTCA).

| Gene Promoter^{a} | MM | MMOCA | MMTCA |
|---|---|---|---|
| Ech3937 (phrpA)^{b} | 41 ± 3 | 82 ± 1 | 78 ± 0 |
| WPP96 (phrpA) | 6.0 ± 0 | 6.5 ± 0.2 | 6.2 ± 0.6 |
| Ech3937 (phrpN) | 43 ± 4 | 94 ± 3 | 103 ± 1 |
| WPP96 (phrpN) | 3.1 ± 0.1 | 3.3 ± 0.1 | 3.3 ± 0 |
| Ech3937 (phrpL) | 14 ± 1 | 19 ± 0 | 19 ± 1 |
| WPP96 (phrpL) | 103 ± 2 | 313 ± 29 | 331 ± 27 |
| Ech3937 (phrpS) | 63 ± 2 | 56 ± 1 | 54 ± 2 |
| WPP96 (phrpS) | 320 ± 60 | 350 ± 17 | 360 ± 16 |
| Ech3937 (pmrp) | 93.5 ± 1.4 | 97.7 ± 1.6 | 105 ± 0.3 |
| Ech3937 (pPROBE-AT) | 1.9 ± 0 | 2.0 ± 0 | 1.9 ± 0 |

^{a}The promoter activities were compared at 12 h of growing in the media. GFP intensity was determined on gated populations of bacterial cells by flow cytometry. The fluorescence intensities were an average GFP fluorescence intensity of total bacterial cells.
^{b}Values (Mean Fluorescence Intensity) are representative of three experiments. Three replicates were used in this experiment. The value is present as the average of three replicates with standard deviation.

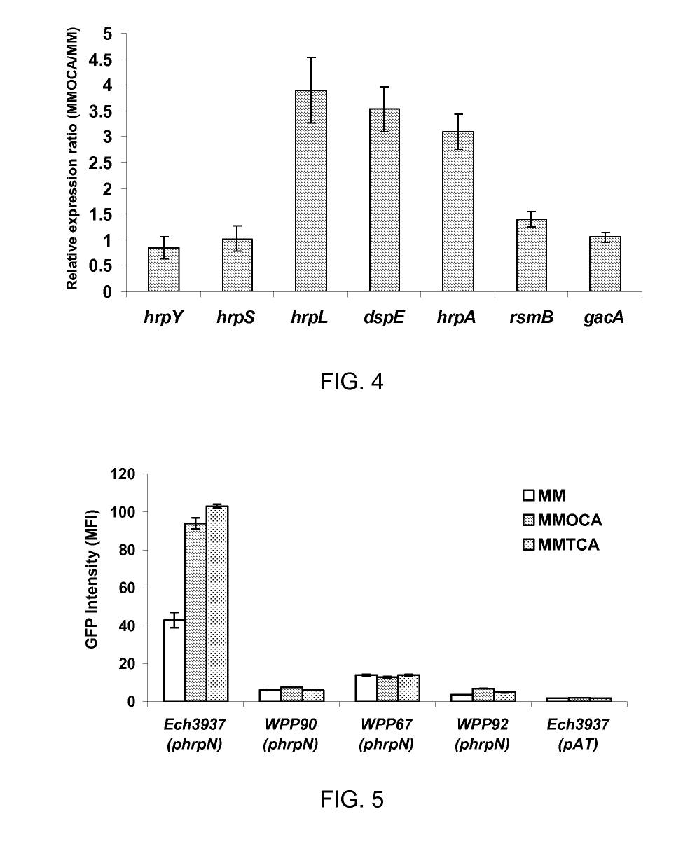

To confirm FACS results showing T3SS induction by plant phenolics, the relative mRNA level of hrpY, hrpS, hrpL, dspE, and hrpA of Ech3937 grown in MM and MM supplemented with OCA was examined by qRT-PCR. Compared with MM alone, a significantly higher amount of dspE and hrpA mRNA was observed in Ech3937 supplemented with OCA (FIG. 4). Although only a slight increase of hrpL promoter activity was observed in Ech3937 (phrpL) grown in MM supplemented with OCA (Table 4), Ech3937 cultures with the supplementation of 0.1 mM OCA produced about 3-fold more hrpL mRNAs than those grown in MM alone at 12 h of growth (P<0.01) (FIG. 4). Three replicates were used in this experiment. The p-value was calculated using Relative Expression Software Tool as described by Pfaffl et al. (2002). No significant difference was found between Ech3937 cells grown in MM and MM supplemented with OCA for gene hrpY, hrpS, and gacA with the p>0.5, but gene expression of hrpL, dspE, hrpA, and rsmB are significantly different between MM and MM supplemented with 0.1 mM OCA with p<0.003 (FIG. 4).

### Example 2

### Regulators Responsible for the OCA and TCA Induction

The expression of T3SS genes dspE, hrpA, and hrpN was reduced in an iaaM mutant Ech138; iaaM encodes an enzyme in the pathway for indole-3-acetic acid (IAA) biosynthesis. To investigate whether IAA biosynthesis is involved in the OCA induction of T3SS, the expression of hrpN in the wild-type Ech3937 and Ech138 was compared with the addition of OCA. As expected, the expression of hrpN was reduced in an iaaM mutant background. However, a similar induction ratio of hrpN by OCA was observed in wild-type Ech3937 and Ech138 at each time point of bacterial growth (Table 5). These results suggest that OCA does not activate T3SS expression through IAA biosynthesis.

TABLE 5
The expression of hrpN of Dickeya dadantii 3937 (Ech3937)
and iaaM mutant Ech138 in MM and MM supplemented with 0.1 mM
OCA (MMOCA), and the expression of hrpA of Ech-Rif and gacA mutant
Ech137 in MM, MM supplemented with 0.1 mM OCA (MMOCA).

| | 6 h | | 12 h | | 24 h | |
|---|---|---|---|---|---|---|
| Gene Promoter^{a} | MM | MMOCA | MM | MMOCA | MM | MMOCA |
| Ech3937 (phrpN)^{b} | 19 ± 2 | 31 ± 3 | 30 ± 3 | 91 ± 8 | 33 ± 2 | 78 ± 8 |
| Ech138 (phrpN) | 5.6 ± 0.7 | 9.1 ± 1.6 | 11 ± 1 | 33 ± 5 | 17 ± 1 | 39 ± 5 |
| Ech-Rif (phrpA) | 13 ± 0 | 18 ± 0 | 22 ± 0 | 51 ± 2 | 16 ± 1 | 49 ± 1 |
| Ech137 (phrpA) | 4.7 ± 0 | 4.9 ± 0.1 | 5.5 ± 0 | 5.9 ± 0.1 | 7.6 ± 0 | 8.3 ± 0 |
| Ech3937 (pPROBE-AT) | 1.9 ± 0 | 2.0 ± 0 | 1.9 ± 0 | 2.0 ± 0 | 1.8 ± 0 | 1.9 ± 0 |

^{a}The promoter activities were compared at 6, 12, and 24 h of bacterial growth. GFP intensity was determined on gated populations of bacterial cells by flow cytometry. The fluorescence intensities were an average GFP fluorescence intensity of total bacterial cells.
^{b}Values (Mean Fluorescence Intensity) are a representative of two experiments. Three replicates were used in this experiment. The value is present as the average of three replicates with standard deviation (SD).

Ech3937 gacA plays a role in regulating the expression of T3SS genes by a post-transcriptional regulation of hrpL through the Gac-Rsm regulatory pathway (Yang, S., Q. Peng, Q. Zhang, X. Yi, C. J. Choi, R. M. Reedy, A. O. Charkowski, and C.-H. Yang. 2008. Dynamic regulation of GacA in type III secretion system, pectinase gene expression, pellicle formation, and pathogenicity of Dickeya dadantii. Mol. Plant-Microbe Interact. 21:133-142.). To investigate whether OCA affects T3SS gene expression through the Gac-Rsm regulatory pathway, the expression of hrpA was compared in wild-type Ech-Rif (phrpA) and gacA mutant Ech137 (phrpA) grown in MM and MM supplemented with OCA, respectively. Compared with Ech-Rif (phrpA) grown in MM alone, a higher GFP intensity was observed in bacterial cells grown in MM supplemented with OCA. However, similar GFP intensity was observed in Ech137 (phrpA) cells grown in MM and MM supplemented with OCA, suggesting that OCA may induce the T3SS gene expression through Gac-Rsm pathway (Table 5). To further confirm that the influence of OCA on T3SS is through the Gac-Rsm system, the expression of gacA and rsmB was examined by qRT-PCR. The results showed that, compared with Ech3937 in MM alone (normalized to 1), a significantly higher rsmB mRNA (relative expression ratio 1.4, P=0.003) was observed in the bacterium grown in MM supplemented with OCA (FIG. 4). The effect of TCA on the mRNA level of rsmB of Ech3937 was also examined. Compared with Ech3937 grown in MM alone (normalized to 1), a significantly higher amount of rsmB mRNA was observed in Ech3937 grown in TCA (1.58, P=0.05). However, no significant difference in the level of gacA mRNA was observed in Ech3937 grown in MM and MM supplemented with OCA (FIG. 4).

HrpL appears to be involved in the induction of T3SS gene expression by the phenolic acids OCA and TCA, as the addition of OCA or TCA did not induce the T3SS gene expression (hrpA and hrpN) in the hrpL mutant background (Table 4). Given that T3SS genes are regulated through HrpX/Y-HrpS-HrpL, experiments were performed to investigate whether OCA and TCA were able to induce the expression of T3SS genes in the absence of hrpX, hrpY, and hrpS, respectively. The GFP intensity of the wild-type Ech3937 and hrpX, hrpY, and hrpS mutants carrying phrpN grown in MM and MM supplemented with OCA and TCA, respectively, was measured (FIG. 5). FIG. 5 shows levels of expression of hrpN of Dickeya dadantii 3937 (Ech3937), hrpS mutant WPP90, hrpX mutant WPP67, hrpY mutant WPP92 in MM, MM supplemented with 0.1 mM OCA (MMOCA), and MM supplemented with 0.1 mM TCA (MMTCA). Ech3937 (pAT) is the wild-type containing the pPROBE-AT vector. The promoter activities were compared at 12 h of growth in the media. GFP intensity was determined on gated populations of bacterial cells by flow cytometry. The fluorescence intensities were an average GFP fluorescence intensity of total bacterial cells. Values (Mean Fluorescence Intensity; MFI) are a representative of three experiments. Three replicates were used in this experiment. The value is present as the average of three replicates with standard deviation (SD). The results showed that OCA and TCA induced the expression of hrpN in Ech3937, but not in hrpX, hrpY, and hrpS mutants (FIG. 5).

### Example 3

### Auto-Regulation of T3SS Regulon

Compared with Ech3937, a higher GFP intensity was observed in the hrpL mutant WPP96 cells carrying phrpS and phrpL, suggesting that HrpL negatively regulated the expression of hrpL and its upstream hrpS (Table 4; FIG. 3). To further confirm the results of GFP-based FACS assays which showed auto-regulation of HrpL, the relative mRNA level of hrpS in Ech3937 and hrpL mutant Ech131 grown in MM was examined by qRT-PCR. Compared with Ech3937, a 4-fold higher level of hrpS mRNA was observed in Ech131 at 6 h of growth in MM (relative expression ratio 5.1, P=0.002). These results suggest that feedback inhibition of HrpL on hrpL itself and hrpS takes place in the bacterium. Similar GFP intensity was observed in Ech3937 and the hrpS mutant carrying phrpS, phrpX and phrpY, respectively, suggesting that HrpS did not regulate the expression of hrpS, hrpX and hrpY. Finally, similar GFP intensity was observed in Ech3937 and the hrpY mutant carrying phrpX and phrpY, respectively, suggesting that HrpY was unable to regulate the expression of hrpX and hrpY.

### Examples 4-9

The Following Methods Apply to Examples 4-9:

Bacterial Strains, Plasmids, and Media.

The bacterial strains and plasmids used in this group of Examiners are listed in Table 6. Wild-type Ech-Rif, and its mutant strains were stored at −80° C. in 15% glycerol and grown in Luria-Bertani (LB) medium (Sambrook, J., and Russell, D. W. 2001. Molecular Cloning: A Laboratory Manual, 3rd ed. Cold Spring Harbor Laboratory, Cold Spring Harbor, N.Y., U.S.A.) and MM medium (Yang et al. 2007). Antibiotics were added to the media at the following concentrations: kanamycin, 50 μg/ml; rifampicin, 100 μg/ml; ampicillin, 100 μg/ml; and spectinomycin, 50 μg/ml. The gacA deletion mutant was constructed by a crossover PCR mutagenesis approach as described (Yang, C. H., Gavilanes-Ruiz, M., Okinaka, Y., Vedel, R., Berthuy, I., Boccara, M., Chen, J. W., Perna, N. T., and Keen, N. T. 2002. hrp genes of Erwinia chrysanthemi 3937 are important virulence factors. Mol. Plant-Microbe Interact. 15:472-480); the primers used were gacA_A, 5′ GCA CCC GAT TGC CTG TAC TTA3′ (SEQ ID NO:19); gacA_B, 5′ GCA CCA GTT CAT GGT CAT CAA C3′ (SEQ ID NO:20); gacA_C, 5′ CGG AGA CAT TGA TTA GTA GTG A3′ (SEQ ID NO:21); and gacA_D, ATT GGG AAA CGG GCC GAA GT (SEQ ID NO:22).

GFP Reporter Plasmid Construction.

The GFP promoter region of dspE and pelD cloned into the reporter plasmid pPROBE-AT (Leveau, J. H., and Lindow, S. E. 2001. Predictive and interpretive simulation of green fluorescent protein expression in reporter bacteria. J. Bacteriol. 183:6752-6762) was constructed previously (Peng et al. 2006). The DNA fragments of promoter regions of hrpA, hrpN, hrpL, and pelL were PCR amplified from Ech3937 chromosomal DNA and ligated into the pCR2.1-TOPO TA cloning vector system (Invitrogen, Carlsbad, Calif., U.S.A.). The primer pair used for pelL promoter in this study is PpelL_F, 5′ATG CGG TAA TGC GGG GAT3′ (SEQ ID NO:23) and PpelL_R, 5′GGC CAG AAC TGA TGT ACT GT3′ (SEQ ID NO:24), which produces a 609-bp pelL promoter region sequence of Ech-Rif. The inserted DNA was further subcloned into the XbaI/SacI sites of the promoter-probe vector pPROBE-AT (Table 6). A plasmid pCLgacA containing a full-length gacA in plasmid pCL1920 also was constructed using the primer set gacAco_F, 5′GCC AAT GTT TCG GGT GTA G3′ (SEQ ID NO:25) and gacAco_R, 5′CAT CGA TCT GCC GGA TAC TTT3′ (SEQ ID NO:26).

The GFP reporter in combination with the FACS-based approach has been used to evaluate gene activity in several bacteria at the single-cell level. Because the gfp gene in the pPROBE-AT contains its own ribosome binding site, the stability of gfp mRNA should not be interfered by RsmA when a promoter-containing DNA region of Ech3937 is cloned into the reporter vector.

FACS Investigation of Promoter Activity.

The bacterial cells of Ech-Rif and Ech137 carrying GFP reporter plasmid constructs were washed three times with 1× phosphate-buffered saline (PBS) buffer (8.0 g of NaCl, 0.2 g of KCl, 1.44 g of Na2HPO4, and 0.24 g of KH2PO4 per liter, pH 7.2 to 7.4) and diluted to approximately 106 CFU/ml before analysis. Bacterial cells were identified based on forward and side light scatter properties and electronically gated for analysis. The promoter activity was determined by FACS (Becton Dickinson, San Jose, Calif., U.S.A.) and the flow cytometry results were analyzed using Cell Quest software (BD Biosciences, San Jose, Calif., U.S.A.).

Real-Time PCR Analysis.

Wild-type Ech-Rif and the gacA mutant Ech137 were grown in MM with glucose as carbon source (Yang et al. 2007). Total RNA from the bacteria was isolated by using TRI reagent method (Sigma-Aldrich, St. Louis, Mo.) and treated with Turbo DNA-free DNase kits (Ambion, Austin, Tex., U.S.A.). An iScript cDNA Synthesis Kit (Bio-Rad, Hercules, Calif., U.S.A.) was used to synthesize cDNA from 0.5 μg of treated total RNA. The Real Master Mix (Eppendorf, Westbury, N.Y., U.S.A.) was used for real-time PCR reaction to quantify the cDNA level of hrpL, rsmA, rsmB, rsmC, and rplU in different samples. The rplU was used as the endogenous control for data analysis. The primer pairs used in this study were RplUsF, 5′ GCG GCA AAA TCA AGG CTG AAG TCG 3′ (SEQ ID NO:27) and RplUsR, 5′ CGG TGG CCA GCC TGC TTA CGG TAG 3′ (SEQ ID NO:28) for rplU; HrpLsF, 5′ GAT GAT GCT GCT GGA TGC CGA TGT 3′ (SEQ ID NO:29) and HrpLsR, 5′ TGC ATC AAC AGC CTG GCG GAG ATA 3′ (SEQ ID NO:30) for hrpL; rsmAf, 5′ TTT TGA CTC GTC GAG TTG GCG AAA 3′ (SEQ ID NO:31) and rsmAr, 5′ GCG CGT TAA CAC CGA TAC GAA CCT 3′ (SEQ ID NO:32) for rsmA; rsmBf, 5′ AGA GGG ATC GCC AGC AAG GAT TGT 3′ (SEQ ID NO:33) and rsmBr, 5′ CGT TTG CAG CAG TCC CGC TAC C3′ (SEQ ID NO:34) for rsmB; and rsmCf, 5′ ACG AAG TGC TCC CGG TTA ATG TCC 3′ (SEQ ID NO:35) and rsmCr, 5′ ACG AGA GCG TAC TGA GCG GCT TTT 3′ (SEQ ID NO:36) for rsmC. Reactions were run and data were collected by the Opticon 2 system (Bio-Rad). Real-time PCR data were analyzed using Relative Expression Software Tool as described (Pfaffl et al., 2002).

Pellicle Formation and Exoenzyme Production.

For pellicle formation assay, bacterial strains were grown in SOBG broth at 28° C. as described (Yap et al. 2005). Due to the slow formation of pellicle in Ech137,10-day-old pellicles from Ech-Rif and Ech137 were used for SEM observation. The samples of pellicle were fixed in 2% glutaraldehyde in PBS buffer (pH 7.0) for 2 h and post-fixed in 1% osmium tetroxide in the same buffer for 1 h. After dehydration in the graded series of ethanol, specimens were infiltrated with polyethylene glycol (PEG). Cross sections of the pellicles were cut using an ultramicrotome. Then, PEG was extracted from the blocks by soaking in several changes of warm ethanol. After critical-point drying, the specimen was mounted on a stub coated with Duco cement, sputter coated with gold, and examined with a Hitachi S-570 Scanning Electron Microscope.

Plate assays for activity of Pel, Cel, and Prt and the spectrophotomeric assay of Pel activity for Ech-Rif, Ech137, and the complemented strain Ech137 (pCLgacA) were carried out as described (Matsumoto, H., Muroi, H., Umehara, M., Yoshitake, Y., and Tsuyumu, S. 2003. Peh production, flagellum synthesis, and virulence reduced in Erwinia carotovora subsp. carotovora by mutation in a homologue of cytR. Mol. Plant-Microbe Interact. 16:389-397). Three biological replicates were performed for each treatment.

Virulence Assay, Growth Kinetics, and in Planta Pel Production.

The local leaf maceration assay was carried out as described (Yang et al. 2004; Yang, C. H., Gavilanes-Ruiz, M., Okinaka, Y., Vedel, R., Berthuy, I., Boccara, M., Chen, J. W., Perna, N. T., and Keen, N. T. 2002. hrp genes of Erwinia chrysanthemi 3937 are important virulence factors. Mol. Plant-Microbe Interact. 15:472-480). Briefly, wild-type bacterial cells and gacA mutant Ech137 cells were syringe-infiltrated in the middle of each symmetric side of the same leaf with approximately 50 μl of a bacterial suspension at 106 CFU/ml. Phosphate buffer (50 mM, pH 7.4) was used to suspend the bacterial cells. Three replicate plants with a total of at least 12 leaves were inoculated. In the systemic invasion assay, the pathogenicity of the bacterium was evaluated as described (Franza, T., Sauvage, C., and Expert, D. 1999. Iron regulation and pathogenicity in Erwinia chrysanthemi 3937: Role of the fur repressor protein. Mol. Plant-Microbe Interact. 12:119-128), with minor modification. A volume of a 50 μl of the bacterial suspension with an optical density at 600 nm of 0.01 was inoculated into the front edge of the African violet leaf. For each bacterial strain, 12 plants were inoculated. Inoculated plants were kept in growth chambers at 28° C., 95% relative humidity, and a photoperiod of 16 h. Development of symptoms induced by bacterial strains in African violet plants was considered as systemic when at least one leaf and its petiole were macerated. Progression of the symptoms was scored daily for 12 days.

Growth kinetics in planta was carried out in African violet cv. Gauguin as described (Yang et al. 2002). Briefly, leaves were syringe infiltrated with approximately 50 μl of bacterial suspension at 10⁶ CFU/ml with a 1-ml syringe. Leaf discs (4 mm in diameter) around the maceration area were harvested at different intervals following infiltration and ground in 50 mM phosphate buffer (pH 7.4). The bacterial concentration, (CFU/cm2) was determined by plating serial dilutions of leaf extracts on LB agar plates. A spectrophotomeric assay was used to monitor the Pel production of Ech-Rif and Ech137 during the in planta growth. A 10-μl supernatant of the plant juice from African violet leaves inoculated with the bacteria was added into 990 μl of Pel reaction buffer and the Pel production was quantified using the spectrophotomeric assay (Matsumoto et al. 2003). Pel production was the ratio of the optical density at 230 nm unit to the log unit of the bacterial concentration (U/log [CFU/cm2]). Three replicate plants with a total of six leaves per plant were used in each sampling time for the in planta Pel production and bacterial growth kinetics assays.

### Example 4

### GacA Affects Biofilm-Pellicle Formation

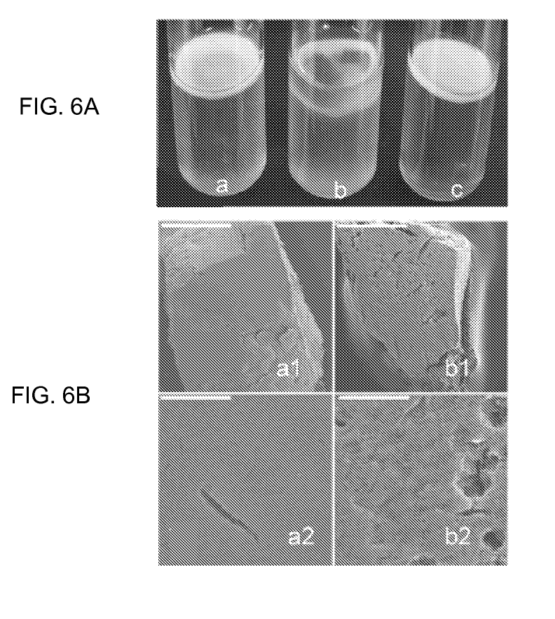

Ech3937 is capable of forming a biofilm and pellicle in SOBG broth. A spontaneous rifampicin-resistant derivative of Ech3937, Ech-Rif, was used as a wild-type in this study (Table 6). A gacA deletion mutant Ech137 of Ech-Rif was constructed and confirmed by DNA sequencing analysis. The gacA gene of Ech137 was deleted with only 20 by of the gacA open reading frame remaining. No significant difference in growth between Ech-Rif and the gacA mutant Ech137 was observed in M9 minimal medium (MM). Ech-Rif formed biofilm-pellicle in SOBG broth grown for 2 days at 28° C. However, no visible biofilm-pellicle was observed in Ech137 until grown for 3 days in SOBG broth. This delayed biofilm-pellicle formation phenotype of the mutant could be restored nearly to the wild-type level when Ech137 was complemented with a low-copy-number plasmid pCLgacA (FIG. 6A). The pellicles of Ech-Rif and Ech137 were sectioned using an ultramicrotome and the interior textures of the pellicles of the bacteria were compared using a scanning electron microscope. A more compact texture in 10-day-old pellicles was observed in Ech-Rif in comparison with Ech137 (FIG. 6B). Pellicles of both Ech-Rif and Ech137 treated with cellulose disintegrated, suggesting that the major component, cellulose, was not altered in Ech137.

FIG. 6A shows biofilm and pellicle formation in SOBG broth (Yap et al. 2005). FIG. 6A(a) shows biofilm and pellicle formed in wild-type Ech-Rif in SOBG cultures grown for 3 days at 28° C. FIG. 6A(b) shows delayed biofilm and pellicle formation in Ech137, the gacA mutant in SOBG cultures grown for 3 days at 28° C. FIG. 6A(c) shows the gacA gene expressed on plasmid pCLgacA restored biofilm and pellicle formation to the gacA mutant Ech137 in SOBG cultures grown for 3 days at 28° C. FIG. 6B shows cross sections of the pellicle observed with scanning electron microscopy at different magnifications. FIG. 6B(a1) shows Ech-Rif; FIG. 6B(b1) shows Ech137; FIG. 6B(a2) shows Ech-Rif; and FIG. 6B(b2) shows Ech137. The size bars in the micrographs in FIG. 6B(a1) and FIG. 6B(b1) are 1 mm, while in FIG. 6B(a2) and FIG. 6B(b2) the size bars are 100 μm.

TABLE 6
Bacterial Strains and Plasmids.

| Strains, plasmids | Characteristics^{a} | Reference or source |
|---|---|---|
| Strains | | |
| Escherichia coli S17-1 λ-pir | λ-pir Lysogen of S17-1 | V. de Lorenzo, Madrid |
| Ech3937 | Wild-type strain isolated from Saintpaulia ionantha | D. Expert, Paris |
| Ech-Rif | Ech3937 rifampicin resistant random mutant | This work |
| Ech137 | ΔgacA::Kan, Km^{r}, Ech-Rif derivative | This work |
| Ech-Rif (pDspE) | Ech3937-Rif containing plasmid pDspE | This work |
| Ech137 (pDspE) | Ech137 containing plasmid pDspE | This work |
| Ech-Rif (pHrpA) | Ech3937-Rif containing plasmid pHrpA | This work |
| Ech137 (pHrpA) | Ech137 containing plasmid pHrpA | This work |
| Ech-Rif (pHrpN) | Ech3937-Rif containing plasmid pHrpN | This work |
| Ech137 (pHrpN) | Ech137 containing plasmid pHrpN | This work |
| Ech-Rif (pHrpL) | Ech3937-Rif containing plasmid pHrpL | This work |
| Ech137 (pHrpL) | Ech137 containing plasmid pHrpL | This work |
| Ech-Rif (pPelD) | Ech3937-Rif containing plasmid pPelD | This work |
| Ech137 (pPelD) | Ech137 containing plasmid pPelD | This work |
| Ech-Rif (pPelL) | Ech3937-Rif containing plasmid pPelL | This work |
| Ech137 (pPelL) | Ech137 containing plasmid pPelL | This work |
| Ech137 (pCLgacA) | Ech137 containing pCLgacA | This work |
| Plasmids | | |
| pWM91 | Sucrose-based counter-selectable plasmid, Ap^{r} | Metcalf et al. 1995 |
| pCR2.1-TOPO | PCR cloning vector, Ap^{r}, Km^{r} | Invitrogen, Carlsbad, CA, |
| | | U.S.A |
| pPROBE-AT | GFP promoter-probe vector, Ap^{r} | Miller et al. 2000 |
| pCL1920 | Low copy number plasmid | Lerner and Inouye 1990 |
| pDspE | pProbe-AT derivative with PCR fragment containing dspE promoter region, Ap^{r} | Yang et al. 2004 |
| pHrpA | pProbe-AT derivative with PCR fragment containing 412-bp hrpA promoter region, Ap^{r} | Unpublished data |
| pHrpN | pProbe-AT derivative with PCR fragment containing 396-bp hrpN promoter region, Ap^{r} | Unpublished data |
| pHrpL | pProbe-AT derivative with PCR fragment containing hrpL promoter region, Ap^{r} | Unpublished data |
| pPelD | pProbe-AT derivative with PCR fragment containing pelD promoter region, Ap^{r} | Peng et al. 2006 |
| pPelL | pProbe-AT derivative with PCR fragment containing 609-bp pelL promoter region, Ap^{r} | This work |
| pCLgacA | pCL1920 with a 1,548-bp PCR product containing full length of gacA, Sp^{r} | This work |

^{a}Ap^{r}, Km^{r}, Rif^{r}, and Sp^{r}, ampicillin, kanamycin, rifampicin, and spectinomycin resistance, respectively; PCR, polymerase chain reaction; and GFP, green fluorescent protein.
Metcalf, W. W., Jiang, W., Daniels, L. L., Kim, S. K., Haldimann, A., and Wanner, B. L. 1996. Conditionally replicative and conjugative plasmids carrying lacZ alpha for cloning, mutagenesis, and allele replacement in bacteria. Plasmid 35: 1-13.
Lerner C. G., and Inouye M. 1990. Low copy number plasmids for regulated low-level expression of cloned genes in Escherichia coli with blue/white insert screening capability. Nucleic Acids Res. 18: 4631.

### Example 5

### GacA Regulates Exoenzyme Production

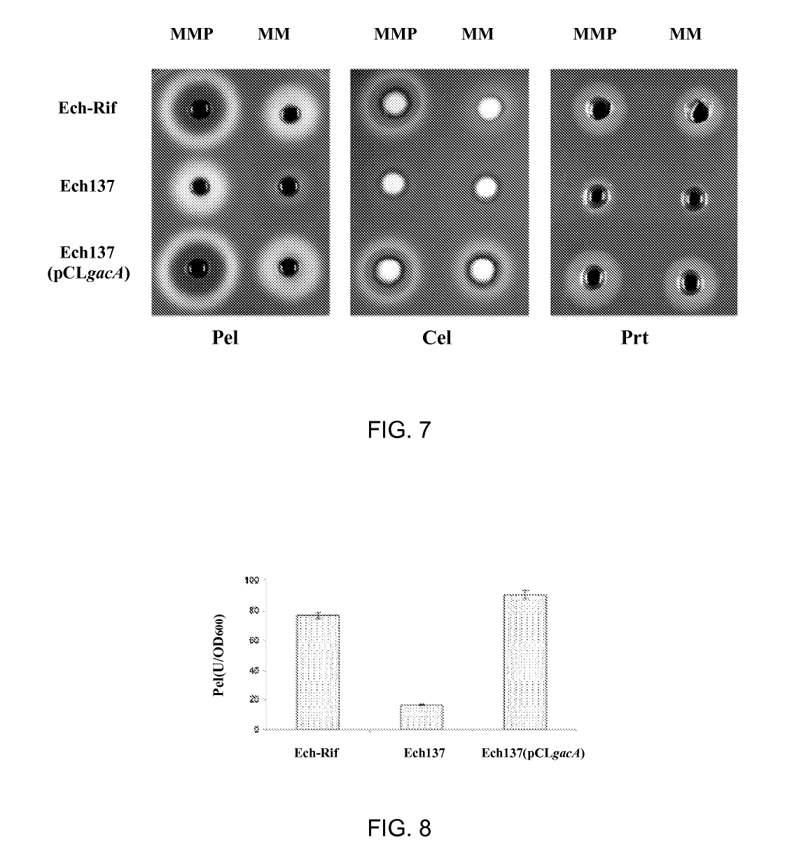

Production of exoenzymes, including pectate lyase (Pel), protease (Prt), and cellulase (Cel) in wild-type Ech-Rif, gacA mutant Ech137, and gacA mutant complemented strain Ech137 (pCLgacA) grown in minimal medium (MM) and MM supplemented with 1% polygalacturonate (MMP) at 36 h, was examined with semi-quantitative plate assays. Values are a representative of two experiments. Three replicates were used in this experiment. Compared with Ech-Rif, reduced Pel, Cel, and Prt production was observed in Ech137 grown in MM and MM supplemented with 1% polygalacturonate (PGA) (MMP) at 36 h (FIG. 7).

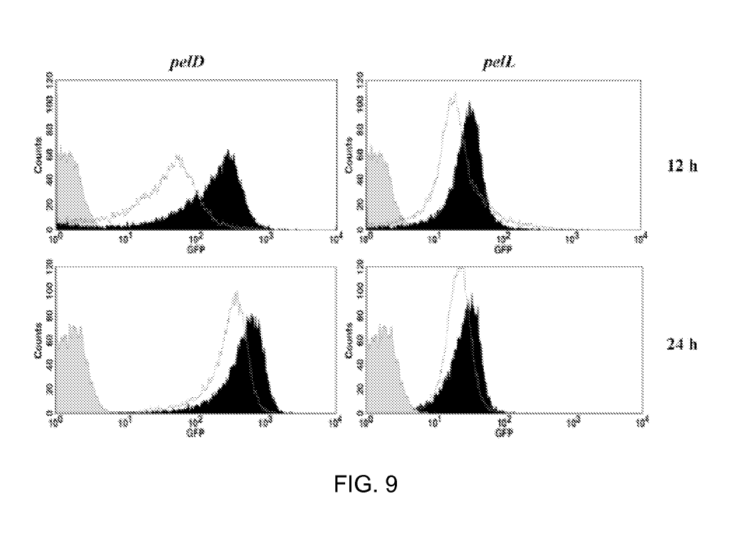

A spectrophotomeric assay was used to quantify Pel activity in the bacterial strains. FIG. 8 shows spectrophotometric quantification of pectate lyase (Pel) activity (U/optical density at 600 nm [OD600]) in Ech-Rif, gacA mutant Ech137, and the complementary strain Ech137 (pCLgacA) grown in minimal medium supplemented with 1% polygalacturonate at 36 h. Values are a representative of two experiments. Three replicates were used in this experiment; the value is present as average of three replicates and the standard deviation. Consistent with the results from the plate assay, lower Pel production by Ech137 was observed by the spectrophotomeric assay when the bacterial cells were grown at 36 h (late stationary phase) (FIG. 8). Compared with the Ech-Rif, a lower Pel production of Ech137 also was observed by the spectrophotomeric assay when the bacterial cells were grown at exponential phase (12 h) and beginning of stationary phase (24 h). The Pel, Cel, and Prt production of Ech137 was restored nearly to the wild-type bacterium level by introducing the plasmid pCLgacA containing wild-type gacA gene into the mutant (FIGS. 7 and 8).

### Example 6

### GacA Regulates the Expression of Pel and T3SS Genes

PelD and PelL of D. dadantii encode endo-Pels. PelD has higher activity on nonmethylated pectins and PelL prefers partially methylated pectins. The promoter regions of pelD and pelL, respectively, were cloned into pPROBE-AT to produce pPelD and pPelL (Table 6). The Ech-Rif and Ech137 cells carrying these GFP promoter plasmids were grown in MM-supplemented 1% PGA and the fluorescence intensity of the bacterial cells was measured with an FACS. The fluorescence intensity collected by FACS was analyzed in a four-decade log scale using CellQuest Pro software from Becton Dickinson, and the gene expression profiles were analyzed as i) total, the average GFP fluorescence intensity of total bacterial cells; ii) GFP+ mean, average GFP fluorescence intensity of GFP expressing bacterial cells; and iii) GFP+%, the percentage of GFP-expressing bacterial cells of the total bacterial cells.

To study the influence of GacA on the transcription of pel genes in Ech3937, the promoter activities of pelD and pelL of Ech-Rif and Ech137 were examined. FIG. 9 shows expression of pelD and pelL in Ech-Rif (black line with black filling) and gacA mutant Ech137 (gray line) grown in minimal medium supplemented with 1% polygalacturonate (MMP). The promoter activities were compared after 12 and 24 h of culture in the medium MMP. Green fluorescent protein (GFP) intensity was determined on gated populations of bacterial cells by flow cytometry and analyzed with the Cell Quest software (BD Biosciences, San Jose, Calif., U.S.A.). The gray line with gray filling stands for the GFP expression control base level of the Ech-Rif containing pPROBE-AT vector without insert. Values are a representative of two experiments. Three replicates were used in this experiment and one replicate was used for the overlay as displayed. Compared with Ech-Rif, a considerably lower expression of pelD and pelL was observed in gacA mutant Ech137 at 12 and 24 h of growth in the medium, indicating that GacA upregulated the expression of pelD and pelL (FIG. 9). The pelD expression in wild-type Ech-Rif is more than twofold higher than that of the gacA mutant Ech137 at 12 h postgrown (FIG. 9). The Ech-Rif cells carrying pPelD at 12 and 24 h were expressed at a mean fluorescence intensity (MFI) of 190±17 and 459±73, while the Ech137 cells carrying pPelD at 12 and 24 h were expressed with an MFI of 52±3 and 324±11. Similarly, the pelL promoter activity in the Ech-Rif cells was approximately 50% greater than that in the gacA mutant Ech137 cells. The MFI values of Ech-Rif (pPelL) were 31±0.1 and 28±0 at 12 and 24 h postgrown, respectively (FIG. 9). The MFI values of Ech137 (pPelL) were 19±1 and 22±1 at 12 and 24 h (FIG. 9).

Regulation of the T3SS genes by GacA has been reported in E. carotovora subsp. carotovora. To study the influence of GacA on the transcription of T3SS genes in Ech3937, the promoter regions of dspE, hrpA, and hrpN of the bacterium were cloned into the pPROBE-AT to produce plasmids pDspE, pHrpA, and pHrpN (Table 6). The total GFP intensity of Ech-Rif (pDspE) was 15±0.8 at 8 h and 17±1.2 at 12 h grown in hrp-inducing MM (Table 7). A lower total GFP intensity of Ech137 (pDspE) of 3.4±0.01 and 3.4±0.05 was observed at the same period of time. Similarly, compared with Ech-Rif, a lower GFP intensity was observed in the Ech137 cells carrying pHrpA and pHrpN grown in the MM at 8 and 12 h (Table 7). The above data provide evidence that GacA upregulated dspE, hrpA, and hrpN.

TABLE 7
Expression of dspE, hrpA, and hrpN of Ech-
Rif and Ech137 grown in minimal medium.

Mean fluorescence intensity^{a}

| Gene promoter | 8 h | 12 h |
|---|---|---|
| Ech-Rif (pDspE) | 15.3 ± 0.8 | 17.0 ± 1.2 |
| Ech137 (pDspE) | 3.4 ± 0.01 | 3.4 ± 0.05 |
| Ech-Rif (pHrpA) | 25.9 ± 1.6 | 39.0 ± 5.0 |
| Ech137 (pHrpA) | 5.4 ± 0.2 | 6.6 ± 0.1 |
| Ech-Rif (pHrpN) | 21.3 ± 1.8 | 38.9 ± 2.4 |
| Ech137 (pHrpN) | 2.6 ± 0.05 | 2.7 ± 0.04 |

^{a}Promoter activities were compared after 8 and 12 h of culture in the minimal medium. Values represent total green fluorescent protein intensity and are a representative of two experiments. Three replicates were used in this experiment. The value is present as average of three replicates and the standard deviation.

### Example 7

### The Gac-Rsm Regulatory Network Controls Pel and T3Ss Gene Expression

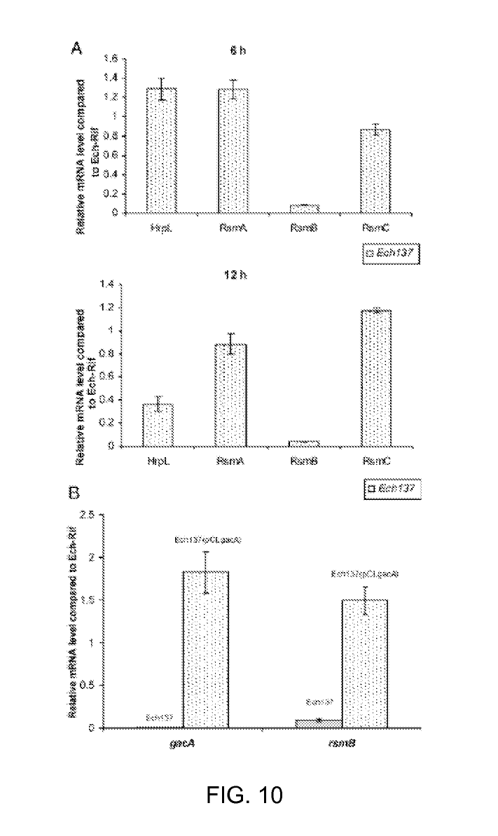

Rsm is a novel type of post-transcriptional regulatory system that plays a critical role in gene expression. To investigate whether the influence of GacA on pectinase gene expression is through the Rsm regulatory pathway, the relative mRNA level of rsmC, rsmB, and rsmA was examined by qRT-PCR. The qRT-PCR data were analyzed using the Relative Expression Software Tool as described by Pfaffl and associates (Pfaffl et al. 2002). FIG. 10A shows relative levels of rsmA, rsmB, rsmC, and hrpL mRNA in gacA mutant Ech137 compared with wild-type Ech-Rif grown for 6 or 12 h in a minimal medium. Three replicates were used in each experiment and the values are presented as the average of the three replicates and the standard deviation. Compared with wild-type, a lower amount of rsmB mRNA was observed in Ech137. Wild-type Ech-Rif produced approximately 10-fold more rsmB mRNA than gacA mutant Ech137 at 6 h and 24-fold more at 12 h, with a P value less than 0.05. No significant differences in amount of rsmC and rsmA mRNA were observed between Ech-Rif and Ech137 (with a P value range from 0.74 to 1) (FIG. 10A).

FIG. 10B shows relative levels of gacA and rsmB mRNA in gacA mutant Ech137 and gacA mutant complemented strain Ech137 (pCLgacA) compared with wild-type Ech-Rif grown for 12 h in a minimal medium. The amount of mRNA was examined by real-time polymerase chain reaction assay and analyzed by Relative Expression Software Tool. The normalized value of mRNA for wild-type was 1.0. Three replicates were used in each experiment and the values are presented as the average of the three replicates and the standard deviation. No detectable mRNA of gacA was observed in Ech137 by qRT-PCR (FIG. 10B). The gacA and rsmB expression of Ech137 was restored by introducing the plasmid pCLgacA into the mutant. The relative mRNA amounts of gacA and rsmB of Ech137 (pCLgacA) are approximately 180 and 150% of the Ech-Rif (FIG. 10B). The higher amounts of gacA and rsmB mRNAs in Ech137 (pCLgacA) compared with Ech-Rif may be due to the copy number effect of the plasmid.

To further investigate whether the influence of GacA on T3SS gene expression is through the GacA-RsmA-rsmB-hrpL regulatory pathway, the amount of hrpL mRNA of the bacteria was examined by qRT-PCR. Compared with Ech-Rif (normalized to 1), a significantly lower hrpL mRNA was observed in gacA mutant Ech137 (0.362±0.065) with a value of 0.001 at 12 h grown in MM (FIG. 10A). A similar amount of hrpL mRNA was observed between Ech-Rif and Ech137 at 6 h (P=1) grown in the medium. Similar promoter activity of hrpL was observed between Ech-Rif and Ech137, suggesting that hrpL was regulated at a post-transcriptional level.

### Example 8

### GacA Influences the Expression of Pel and T3SS Genes in Planta

To connect the in vitro result with the in vivo condition, the expression of pelL and dspE between Ech-Rif and Ech137 in host plant African violet (Saintpaulia ionantha) leaves was examined further. Compared with Ech137, a higher transcription of pelL and dspE in Ech-Rif in S. ionantha was observed at 24 h postinoculation, which is approximately threefold more for pelL and fourfold more for dspE (Table 8).

TABLE 8
Expression of pelL and dspE of Ech-Rif and
gacA mutant Ech137 in African violet^{a}

| Gene | Ech-Rif | Ech137 | Ech-Rif | Ech137 |
|---|---|---|---|---|
| promoter | (pPelL) | (pPelL) | (pDspE) | (pDspE) |
| Total | 51.4 ± 20.91 | 11.8 ± 7.4 | 6.0 ± 0.8 | 1.2 ± 0.1 |
| GFP⁺ mean | 78.2 ± 17.0 | 40.1 ± 0.7 | 54.4 ± 12.2 | 15.8 ± 15.2 |
| GFP⁺% | 57.1 ± 14.9 | 11.6 ± 11.3 | 8.0 ± 0.5 | 0 ± 0 |

^{a}Promoter activities were compared after 24 h of inoculation. Green fluorescent protein (GFP) intensity was determined on gated populations of bacterial cells by flow cytometry. Values are a representative of two experiments. Three replicates were used in this experiment. The value is present as average of three replicates and the standard deviation.

### Example 9

### The gacA Mutant Reduced Maceration and Systemic Invasion Ability

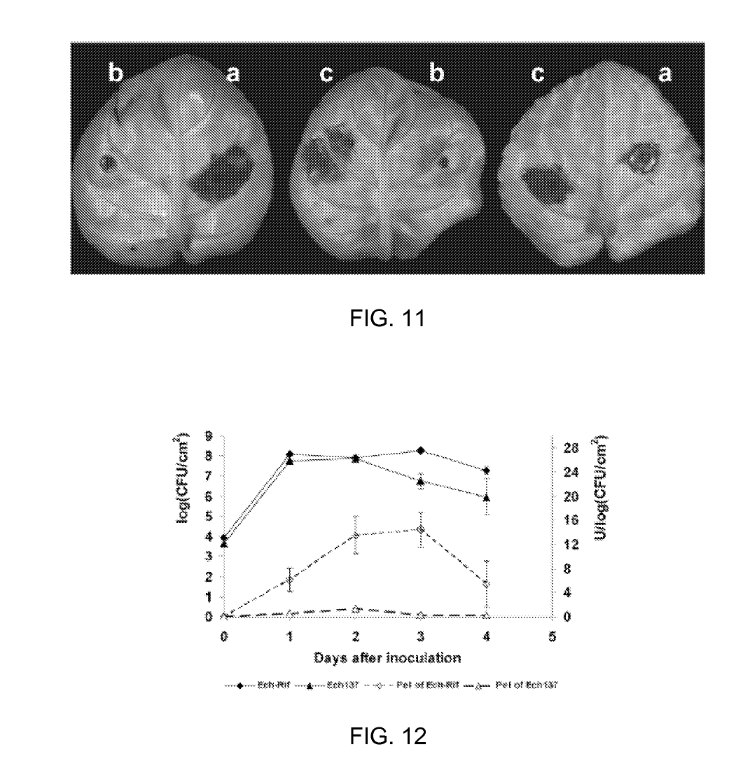

Because GacA affects multiple phenotypes contributing to pathogenesis, a local maceration assay was carried out with Ech-Rif, Ech137, and the complemented strain Ech137 (pCLgacA) in the African violet cv. Gauguin as previously described (Yang et al. 2002). FIG. 11 shows local maceration lesions caused by a, Ech-Rif; b, gacA mutant Ech137; and c, complemented strain Ech137 (pCLgacA). Bacterial cells were inoculated in the middle of each half side of the same leaf. Phosphate buffer (pH 7.4, 50 mM) was used to suspend the bacterial cells and a volume of a 50 μl of bacterial suspension with a bacterial concentration of 106 CFU/ml was used. The maceration symptom was examined 2 days postinoculation. The experiment has been repeated twice. Compared with Ech-Rif, Ech137 was dramatically reduced in maceration ability in planta 2 days postinoculation (FIG. 11). The maceration ability of Ech137 was restored to near the wild-type Ech-Rif level by pCLgacA (FIG. 11).

FIG. 12 shows the concentration of Ech-Rif and gacA mutant Ech137 in African violet cv. Gauguin (Saintpaulia ionantha). Leaves of African violet were inoculated with a 50-μl bacterial suspension at a concentration of 10⁶ CFU/ml. Six leaves from six replicate plants were used at each sampling time for each bacterial strain, the value is present as average of three replicates and the standard deviation; concentration of Ech-Rif (solid diamonds) and Ech137 (solid triangles). The spectrophotometric quantification was carried out as described to measure the pectinase (Pel) activity in the inoculated leaves from the same sample for population kinetics; Pel production of Ech-Rif (open diamonds) and Ech137 (open triangles). Interestingly, although a lower Pel activity also was observed in plant leaves inoculated with Ech137, in comparison with the leaves inoculated with wild-type Ech-Rif (FIG. 12), there was no difference in bacterial concentration between the wild-type Ech-Rif and Ech137 in African violet leaves at day 2 postinoculation analyzed by a paired sample t test (P=0.23) (FIG. 12). Compared with Ech-Rif, a lower bacterial concentration of Ech137 in plants was observed at day 3 (P=5.9×10⁻¹¹) and day 4 (P=1.7×10⁻⁴).

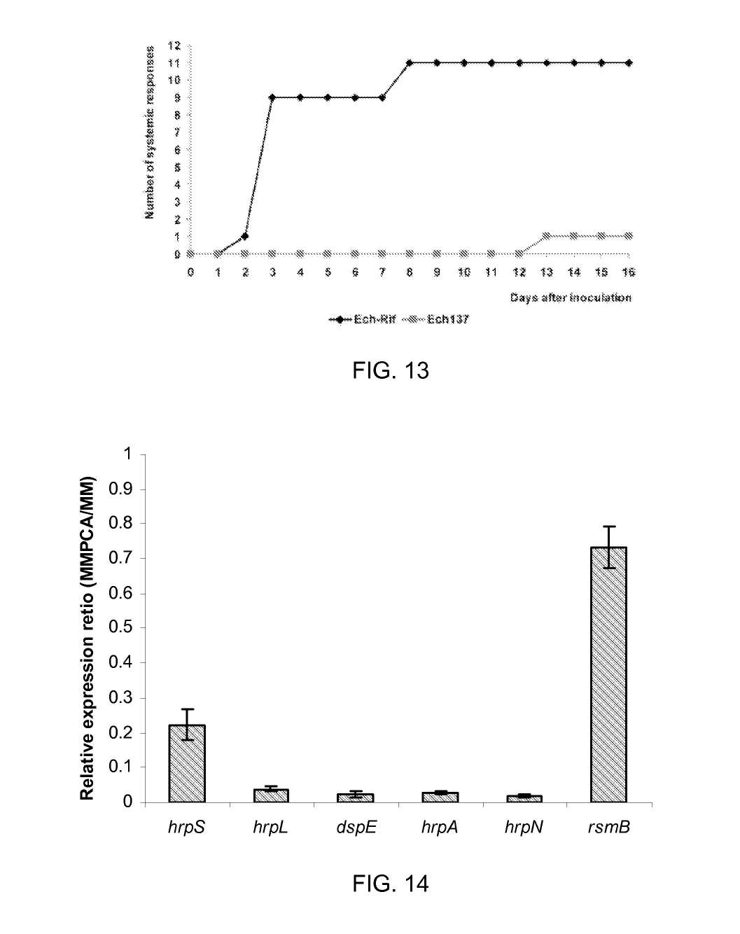

A systemic invasion assay (Franza et al. 1999) was further applied to investigate the role of GacA of the bacterium in S. ionantha. For each bacterial strain (Ech-Rif and Ech137), 12 plants (one leaf per plant) were inoculated. Response was considered as systemic when at least one leaf and its petiole were macerated. Values are a representative of two experiments. Eight days after inoculation, 11 of the 12 plants inoculated with Ech-Rif developed systemic invasion symptoms (FIG. 13). In contrast, the Ech137 showed a reduced ability to develop a systemic invasion in the plant host; only one plant developed a systemic invasion with the gacA mutant 16 days postinoculation.

### Example 10

### Identification of T3SS Inhibitors

To identify potential compounds that repress activation of T3SS, analogs and isomers of TCA and intermediates of salicylic acid and phenypropanoid biosynthesis pathways in plants were further screened for their effect on Ech3937 hrpA expression by flow cytometry (Table 9). A green fluorescent protein (GFP) reporter plasmid phrpA (pProbe-AT derivative with PCR fragment containing hrpA promoter region) was constructed, resulting in a transcriptional fusion of hrpA promoter with GFP gene. Bacterial cells containing phrpA plasmid were grown in MM supplemented with 0.1 mM of each compound. GFP intensity, which is a measurement of hrpA promoter activity, was measured by flow cytometry. The TCA analogs p-coumaric acid (PCA) and cinnamyl alcohol were discovered to be inhibitors of the expression of hrpA (Table 9).

TABLE 9
The expression of hrpA of Dickeya dadantii 3937
(Ech3937) in MM and MM supplemented with different
isomers and analogs of t-cinnamic acid.

| Phenolic compound^{a} | 6 h^{b} | 12 h | 24 h |
|---|---|---|---|
| MM | 8.3 ± 0.7 | 78.7 ± 6.3 | 92.1 ± 17.1 |
| t-Cinnamic acid | 22.3 ± 2.1 | 133.9 ± 12.9 | 203.7 ± 16.1 |
| o-Coumaric acid | 15.5 ± 1.1 | 115.5 ± 7.9 | 225.8 ± 15.6 |
| m-Coumaric acid | 12.1 ± 0.0 | 133.0 ± 38.2 | 203.3 ± 9.6 |
| p-Coumaric acid | 6.9 ± 0.1 | 10.2 ± 0.4 | 11.4 ± 1.0 |
| Hydrocinnamic acid | 13.7 ± 1.0 | 200.3 ± 35.8 | 213.5 ± 18.9 |
| Phenoxyacetic acid | 18.0 ± 3.1 | 222.7 ± 64.3 | 205.7 ± 11.8 |
| trans-2-Phenylcyclopropane- | 7.2 ± 0.1 | 67.0 ± 18.4 | 84.0 ± 14.3 |
| 1-carboxylic acid | | | |
| trans-3-(3-Pyridyl)acrylic | 13.1 ± 0.4 | 184.9 ± 35.6 | 204.0 ± 16.8 |
| acid | | | |
| trans-3-indoleacrylic acid | 7.9 ± 0.2 | 23.9 ± 1.3 | 121.0 ± 6.2 |
| 2-Methylcinnamic acid | 22.8 ± 0.6 | 157.2 ± 11.7 | 342.5 ± 16.6 |
| 2-Chlorocinnamic acid | 25.7 ± 0.2 | 166.2 ± 17.8 | 319.8 ± 48.3 |
| Methyl trans-cinnamate | 9.7 ± 0.1 | 135.8 ± 8.1 | 219.2 ± 14.5 |
| Cinnamyl alcohol | 6.3 ± 0.1 | 27.8 ± 5.0 | 53.8 ± 2.9 |

^{a}Minimum medium (MM) and MM supplemented with 0.1 mM of different compounds.
^{b}Ech3937 cells carrying GFP reporter phrpA was used in this study. The promoter activities were compared at 6, 12, and 24 h of bacterial growth. GFP intensity was determined on gated populations of bacterial cells by flow cytometry. The fluorescence intensities were an average GFP fluorescence intensity of total bacterial cells. Values (Mean Fluorescence Intensity) of GFP are a representative of three experiments. Three replicates were used in this experiment. The value is present as the average of three replicates with standard deviation (SD).

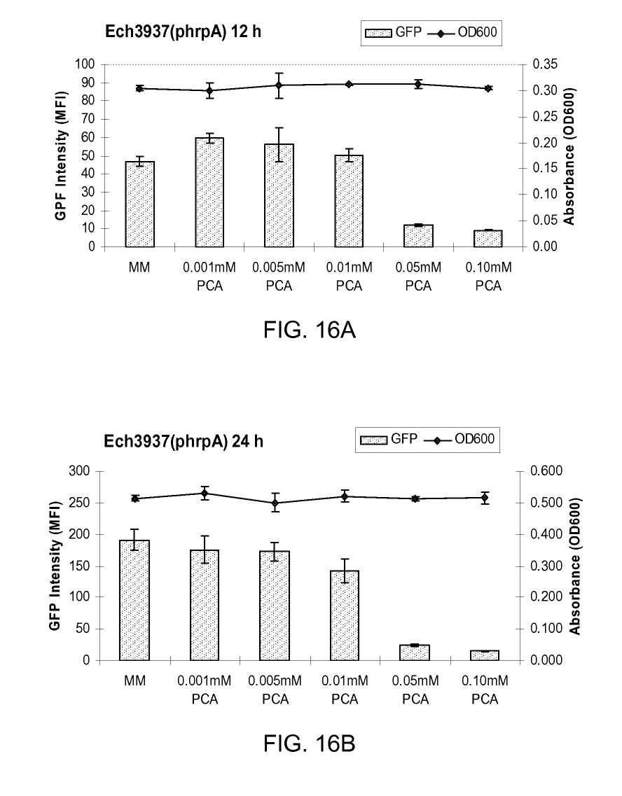

The expression of the T3SS gene hrpA was further examined in MM and MM supplemented with PCA at concentrations of 0.001, 0.005, 0.01, 0.05 and 0.1 mM respectively. FIGS. 16A and 16B show the promoter activities of hrpA in Dickeya dadantii 3937 (Ech3937) grown in minimum medium (MM) and MM supplemented with different amount of p-coumaric acid (PCA) at 12 h (FIG. 16A) and 24 h (FIG. 16B) post-inoculation. GFP intensity was determined on gated populations of bacterial cells by flow cytometry and analyzed with the Cell Quest software (BD Biosciences, San Jose, Calif.). The growth of Ech3937 in MM supplemented with different concentrations of PCA was recorded. Compared with MM alone, the average GFP fluorescence intensity of bacterial cells of Ech3937 (phrpA) was reduced more than 4-fold when 0.05 and 0.1 mM of PCA were added to the medium (FIGS. 16A, 16B). The addition of PCA at concentrations at 0.001, 0.005 and 0.01 did not result in substantial reduction of GFP fluorescence intensity of Ech3937. Inhibition of bacterial growth was not observed when PCA was added into the MM (FIGS. 16A, 16B).

To confirm the inhibitory effect of PCA on T3SS of Ech3937, the promoter activity of hrpN of Ech3937 was also examined. A reduced expression of hrpN was observed in MM supplemented with 0.1 mM PCA in comparison with the bacterial cells grown in MM alone (Table 10). The mrp, whose protein product has an ATPase conserved domain (2e-06), was used as a reference gene. Similar mrp expression was observed in Ech3937 (pmrp) when the bacterial cells were grown in MM and MM supplemented with 0.1 mM PCA respectively (Table 10). The repression effect of PCA on T3SS gene expression was further demonstrated by a qRT-PCR assay. FIG. 14 shows the relative mRNA level of hrpS, hrpL, dspE, hrpA, hrpN, and rsmB of Dickeya dadantii 3937 (Ech3937) in minimum medium (MM) supplemented with 0.1 mM p-coumaric acid (PCA) compared to those in MM without PCA. The amount of mRNA was determined by qRT-PCR. Three replicates were used in this experiment. The p-value was calculated using Relative Expression Software Tool as described by Pfaffl et al. (2002). Levels of gene expression of hrpS, hrpL, dspE, hrpA, and hrpN are significantly different between MM and MM supplemented with 0.1 mM PCA with p<0.001. Compared with Ech3937 in MM alone (normalized to 1), a significantly lower amount of mRNA of dspE (P=0.001), hrpA (P=0.001), and hrpN(P=0.001) was observed in the bacterium grown in MM supplemented with PCA (FIG. 14).

TABLE 10
The expression of type III secretion genes hrpA and hrpN
of Dickeya dadantii 3937 (Ech3937) in minimum medium
(MM) and MM supplemented with 0.1 mM PCA (MMPCA).

| | 12 h | | 24 h | |
|---|---|---|---|---|
| Gene Promoter^{a} | MM | MMPCA | MM | MMPCA |
| Ech3937 (phrpA) | 64.3 ± 0.9^{b} | 10.3 ± 1.3 | 150.8 ± 4.4 | 18.5 ± 3.5 |
| Ech3937 (phrpN) | 39.8 ± 5.8 | 6.8 ± 0.8 | 133.3 ± 3.2 | 11.3 ± 3.4 |
| Ech3937 (phrpS) | 72.7 ± 11.3 | 37.9 ± 1.3 | 95.0 ± 17.7 | 43.6 ± 2.6 |
| Ech3937 (phrpL) | 12.8 ± 0.1 | 7.8 ± 0.1 | 27.2 ± 1.0 | 11.1 ± 3.3 |
| Ech3937 (pmrp) | 113.0 ± 7.7 | 124.1 ± 2.7 | 93.4 ± 2.6 | 98.9 ± 1.0 |
| Ech3937 (pPROBE-AT) | 2.1 ± 0.1 | 2.2 ± 0.2 | 13.4 ± 8.4 | 14.0 ± 10.1 |

^{a}The promoter activities were compared at 12 and 24 h of bacterial growth in p-coumaric acid (PCA).
^{b}GFP intensity was determined on gated populations of bacterial cells by flow cytometry. The fluorescence intensities were an average GFP fluorescence intensity of total bacterial cells. Values (Mean Fluorescence Intensity) are a representative of two experiments. Three replicates were used in this experiment. The value is present as the average of three replicates with standard deviation (SD).

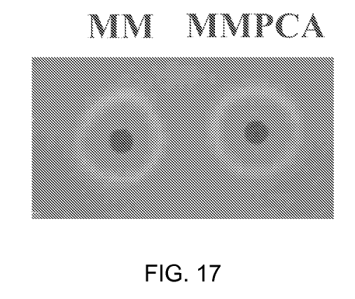

GacS/GacA also induced the production of pectate lyase of Ech3937. To investigate whether PCA affects the T3SS gene expression through the Gac-Rsm regulatory pathway, the expression of rsmB was further examined by qRT-PCR. FIG. 17 shows pectate lyase (Pel) production of Dickeya dadantii 3937 (Ech3937) grown in minimal medium (MM) and MM supplemented with 0.1 mM of p-coumaric acid (PCA) at 12 h examined by plate assays as described (Matsumoto et al. 2003). Values are a representative of two experiments. Three replicates were used in this experiment. No significant difference was observed between Ech3937 grown in MM and MM supplemented with PCA for gene rsmB with the p=0.928 (FIG. 14). This result shows that the repression T3SS expression by PCA is not through the Gac-Rsm pathway. That the Gac-Rsm pathway is not interfered by PCA is further suggested by a pectinase assay. Similar pectate lyase production was observed between Ech3937 grown in MM and MM supplemented with 0.1 mM PCA, demonstrating that GacS/GacA is not influenced by PCA (FIG. 17).

Along with GacS/GacA-RsmA-rsmB-hrpL regulatory pathway, the T3SS of Ech3937, which belongs to Group I T3SS of phytobacteria, is primarily regulated by a HrpX/Y-HrpS-HrpL pathway. The two-component system HrpX/HrpY activates the gene encoding HrpS, which is required for expression of hrpL. HrpL, an alternative sigma factor, further activates expression of genes encoding the T3SS apparatus and its secreted substrates. To investigate whether PCA represses T3SS gene expression through HrpX/Y-HrpS-HrpL pathway, the promoter activity of hrpS and hrpL was further examined. Compared with Ech3937(phrpS) and Ech3937(phrpL) in MM, a lower GFP intensity was observed in the bacterial cells carrying phrpS and phrpL grown in MM supplemented with 0.1 mM PCA (Table 10). The expression of hrpS and hrpL was also confirmed by qRT-PCR. The result showed that, compared with Ech3937 in MM alone (normalized to 1), a significantly lower amount of mRNA of hrpS (relative expression ratio 0.223, P=0.001) and hrpL (relative expression ratio 0.039, P=0.001) was observed in bacteria grown in MM supplemented with PCA (FIG. 14).

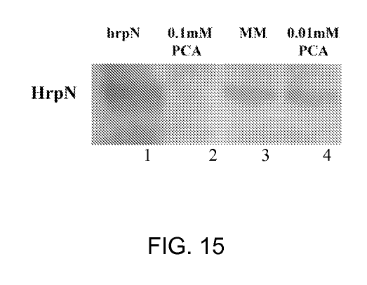

To further elucidate whether T3SS gene expression is mainly regulated through the HrpX/Y-HrpS-HrpL regulatory pathway, the expression of hrpA and hrpN in wild-type Ech3937 and hrpX (WPP67), hrpY (WPP92), hrpS (WPP90), and hrpL (WPP96) mutants in MM were examined. The result showed that compared with Ech3937 carrying phrpA and phrpN, very low expression of hrpA and hrpN was observed in WPP67, WPP92, WPP90, and WPP96 grown in MM. Similar expression of hrpA and hrpN was observed among WPP67, WPP92, WPP90 and WPP96 carrying phrpA and phrpN grown in MM and MM supplemented with PCA. These results demonstrate that hrpX, hrpY, hrpS and hrpL are crucial for the T3SS gene expression and PCA inhibits expression of T3SS genes through HrpX/Y-HrpS-HrpL regulatory pathway (Table 11). Since PCA represses the expression of several T3SS genes such as hrpN, the effect of PCA on protein production of HrpN was further examined. FIG. 15 shows HrpN protein expression of Dickeya dadantii 3937 (Ech3937) in minimum medium (MM) and MM supplemented with 0.1 mM of p-Coumaric acid (PCA). Lane 1 of FIG. 15 shows a HrpN overexpression strain. Lane 2 shows Ech3937 grown in MM supplemented with 0.1 mM PCA. Lane 3 shows Ech3937 grown in MM. Lane 4 shows Ech3937 grown in MM supplemented with 0.01 mM PCA. Compared with MM alone, a lower amount of HrpN was observed in Ech3937 grown in MM supplemented with 0.1 mM of PCA (FIG. 15).

TABLE 11
The expression of hrpA and hrpN of wild-type Dickeya dadantii 3937 (Ech3937)
and hrpX (WPP67), hrpY (WPP92), hrpS (WPP90) and hrpL (WPP96) mutants in
minimum medium (MM) and MM supplemented with 0.1 mM PCA (MMPCA).

| | 12 h | | 24 h | |
|---|---|---|---|---|
| Gene Promoter^{a} | MM | MMPCA | MM | MMPCA |
| Ech3937 (phrpA) | 53.0 ± 5.8^{b} | 9.2 ± 0.4 | 139.3 ± 22.2 | 12.7 ± 0.3 |
| WPP67 (phrpA) | 8.3 ± 0.1 | 7.9 ± 0.2 | 10.3 ± 1.1 | 9.0 ± 1.3 |
| WPP92 (phrpA) | 9.3 ± 0.2 | 6.9 ± 0.1 | 9.1 ± 0.2 | 9.4 ± 0.7 |
| WPP90 (phrpA) | 7.7 ± 0.4 | 7.9 ± 0.2 | 8.1 ± 0.0 | 9.3 ± 0.2 |
| WPP96 (phrpA) | 7.6 ± 0.3 | 7.3 ± 0.1 | 7.6 ± 0.1 | 9.0 ± 1.6 |
| Ech3937 (phrpN) | 49.0 ± 3.3 | 4.3 ± 0.1 | 112.2 ± 6.7 | 8.8 ± 2.1 |
| WPP67 (phrpN) | 4.4 ± 0.4 | 3.5 ± 0.2 | 9.4 ± 1.4 | 6.3 ± 0.7 |
| WPP92 (phrpN) | 3.8 ± 0.1 | 3.6 ± 0.1 | 5.7 ± 0.4 | 5.5 ± 0.3 |
| WPP90 (phrpN) | 3.6 ± 0.2 | 3.5 ± 0.1 | 5.0 ± 0.3 | 5.1 ± 0.1 |
| WPP96 (phrpN) | 3.0 ± 0.0 | 3.0 ± 0.0 | 3.9 ± 0.1 | 4.3 ± 0.1 |
| Ech3937 (Pprobe-AT) | 2.2 ± 0.1 | 2.3 ± 0.1 | 4.3 ± 0.2 | 4.9 ± 0.1 |

^{a}The promoter activities were compared at 12 and 24 h of bacterial growth in p-Coumaric acid (PCA).
^{b}GFP intensity was determined on gated populations of bacterial cells by flow cytometry. The fluorescence intensities were an average GFP fluorescence intensity of total bacterial cells. Values (Mean Fluorescence Intensity) are a representative of two experiments. Three replicates were used in this experiment. The value is present as the average of three replicates with standard deviation (SD).

### Example 11

### Screening of Compounds for Inhibition of Bacterial Virulence

Once phenylalanine derivatives have been produced, each will be tested for its efficacy in the reduction of bacterial virulence. Changes in bacterial virulence will be assessed by monitoring changes in activities of a GacS/GacA-type system, a HrpX/HrpY-type system, an Rsm system, a T3SS system, or downstream genes, gene products, and other effectors, as described herein.

Various assays will be used to test the efficacy of the phenylalanine derivatives, such as any of the assays described herein, including without limitation promoter-probe bioreporter assays, cell sorting (FACS), pectinase activity assays, qRT-PCR analysis, analysis of the phosphorylation of GacA or HrpY, leaf maceraction assays, growth kinetics assays, plate assays, analysis of pellicle formation, analysis of exoenzyme production, and spectrophotometric quantification assays. Assays will be performed using Dickeya dadantii or any other suitable bacterial species or strain having a GacS/GacA-type system, a HrpX/HrpY-type system, an Rsm-type system, and/or a T3SS-type system. Leaf maceraction assays will be carried out using leaves from any of a variety of plants, including African violet, Chinese cabbage, or witloof chicory leaves.

The results of the assays will be evaluated in accordance with the methods described herein and according to the knowledge of one skilled in the art to determine whether the compound that is tested reduces bacterial virulence. Those compounds that demonstrate activity in reducing bacterial virulence in the assays will then be used as antimicrobial compounds, for example as described in the subsequent Examples.

### Example 12

### Use on Plants

The active compounds, compositions containing the active compounds, and methods of using the same, will be used with any plant including those having an appropriate TCSTS-containing bacterium associated therewith, including a bacterium having a GacS/GacA-type system or a HrpX/HrpY-type system. These plants will include cultivated, domesticated, or wild plants, including annual crops and longer-term crops such as trees. Agriculturally relevant annual crops include, without limitation, corn, soy, wheat, barley, oats, rice, sorghum, rye, alfalfa, tobacco, and sunflower. The compounds, compositions, and methods will be used either on terrestrial plants or on aquatic plants, including freshwater and marine-dwelling plants.

For application on plants, the compounds will be formulated in compositions such as a liquid suitable for application by spraying or other mode of application; dust; granules; oil; or solid (e.g. as a spike). The composition will be produced in a concentrated form, including a concentrated liquid, powder, solid, or other form, which will be reconstituted prior to use.

The following are suitable as possible formulations: wettable powders (WP), water-soluble powders (SP), emulsifiable concentrates (EC), aqueous solutions or concentrates, emulsions (EW), sprayable solutions, capsule suspensions (CS), dispersions on an oil or water base, suspoemulsions, suspension concentrates (SC), dusting powders (DP), solutions which can be mixed with oils (OL), seed-dressing agents, granules (GR) in the form of microgranules, spray granules, coated granules and adsorption granules, granules for broadcasting and soil application, water-soluble granules (SG), water-dispersible granules (WG), ULV formulations, microcapsules and waxes.

These individual types of formulations are known in principle and are described, for example, in: Winnacker-Kuchler, “Chemische Technologie [Chemical Technology]”, Volume 7, C. Hauser Verlag Munich, 4th Ed. 1986; van Valkenburg, “Pesticides Formulations”, Marcel Dekker N.Y., 2nd Ed. 1972-73; K. Martens, “Spray Drying Handbook”, 3rd Ed. 1979, G. Goodwin Ltd. London.

The formulation auxiliaries required, such as inert materials, surfactants, solvents and other additives are likewise known and are described, for example, in: Watkins, “Handbook of Insecticide Dust Diluents and Carriers”, 2nd Ed., Darland Books, Caldwell N.J.; H. V. Olphen, “Introduction to Clay Colloid Chemistry”; 2nd Ed., J. Wiley & Sons, N.Y., Marsden, “Solvents Guide”, 2nd Ed., Interscience, N.Y. 1950; McCutcheon's, “Detergents and Emulsifiers Annual”, MC Publ. Corp., Ridgewood N.J.; Sisley and Wood, “Encyclopedia of Surface Active Agents”, Chem. Publ. Co. Inc., N.Y. 1964; Schonfeldt, “Grenzflachenaktive Athylenoxidaddukte [Surface-active Ethylene Oxide Adducts]”, Wiss. Verlagsgesell., Stuttgart 1976; and Winnacker-Kuchler, “Chemische Technologie [Chemical Technology]”, Volume 7, C. Hauser Verlag Munich, 4th Ed. 1986.

Combinations with other active substances such as herbicides, fungicides or insecticides, as well as fertilizers and/or growth regulators, will also be prepared on the basis of these formulations, for example in the form of a ready-mix or as a tank mix.

The active compound combinations according to the invention can either be a mixed formulation of the two components which are then diluted with water and applied in a customary manner, or they can be prepared as so-called tank mixes by joint dilution, with water, of the separately formulated components.

Wettable powders are preparations which are uniformly dispersible in water and which, besides the active compound, also contain wetting agents, for example polyoxethylated alkylphenols, polyoxethylated fatty alcohols or fatty amines, alkane- or alkylbenzenesulfonates, and dispersing agents, for example sodium ligninsulfonate, sodium 2,2′-dinaphthylmethane-6,6′-disulfonate, sodium dibutylnaphthalenesulfonate, or alternatively sodium oleylmethyltaurinate, in addition to a diluent or inert substance.

Emulsifiable concentrates will be prepared by dissolving the active compound in an organic solvent, for example butanol, cyclohexanone, dimethylformamide, xylene and also higher-boiling aromatic compounds or hydrocarbons, with the addition of one or more emulsifiers. Examples of emulsifiers which may be used are: calcium salts of an alkylarylsulfonic acid, such as Ca dodecylbenzenesulfonate, or non-ionic emulsifiers, such as fatty acid polyglycol esters, alkylaryl polyglycol ethers, fatty alcohol polyglycol ethers, propylene oxide/ethylene oxide condensation products, alkyl polyethers, sorbitan fatty acid esters, polyoxyethylene sorbitan fatty acid esters or polyoxyethylene sorbitol esters.

Dusting agents will be obtained by grinding the active compound with finely divided solid substances, for example talc or natural clays, such as kaolin, bentonite, pyrophillite or diatomaceous earth.

Granules may be produced either by spraying the active substance onto adsorptive, granulated inert material or by applying active substance concentrates onto the surface of carriers, such as sand, kaolinites or of granulated inert material, by means of binders, for example polyvinyl alcohol, sodium polyacrylate or, alternatively, mineral oils. Suitable active substances may also be granulated in the manner which is conventional for the production of fertilizer granules, if desired in a mixture with fertilizers.

In one embodiment, the concentration of active compound in wettable powders will be, for example, about 10 to about 95% by weight, the remainder to 100% by weight is composed of conventional formulation components. In the case of emulsifiable concentrates, the concentration of active compound may be about 1 to about 85% by weight, preferably about 5 to about 80% by weight. Formulations in the form of dusts usually contain about 1 to about 25% by weight, mostly about 5 to about 20% by weight of active compound, sprayable solutions about 0.2 to about 25% by weight, preferably about 2 to about 20% by weight, of active compound. In the case of granules, such as water-dispersible granules, the active compound content depends partly on whether the active compound is liquid or solid and on which granulation auxiliaries and fillers are used. The content in water-dispersible granules is generally between about 10 and about 90% by weight.

In addition, the active compound formulations mentioned contain, if appropriate, the adhesives, wetting agents, dispersing agents, emulsifiers, penetrants, solvents, fillers or carriers which are conventional in each case.

For use, the formulations, present in commercially-available form, are diluted, if appropriate, in a customary manner, for example using water in the case of wettable powders, emulsifiable concentrates, dispersions and water-dispersible granules. Preparations in the form of dusts, granules for soil application and/or broadcasting, and also sprayable solutions are usually not further diluted with other inert substances before use.

The application rate required for the compositions varies with the external conditions, such as, inter alia, temperature, humidity, and the nature of the compound and composition used.

Further information regarding formulation and application of compositions for agricultural use are disclosed in U.S. Pat. No. 5,447,903, incorporated herein by reference.

The compositions will be applied to different parts of the plant, including leaves, stems, roots, buds, and fruits, as well as to soil in the vicinity of a plant. Methods of application will include spraying, irrigating, dusting, and spreading or broadcasting of granules, powders, or other solid or liquid forms.

The composition will be applied to leaf surfaces, stems of plants including agricultural crops, and irrigated into soil to protect root systems from human and other pathogens (including, for example, E. coli O157:H7 on lettuce, spinach) which may be present as contaminants from exposure to animal waste.

The composition will be applied to the surfaces of stored crops (including without limitation onions, potatoes, grains, squash, melons) to reduce post-harvest infection and contamination by plant, animal, and human pathogens.

The composition will be applied to leaf surfaces, stems, fruits, and other portions of plants intended for consumption by animals including humans, either for fresh consumption or consumption following cooking or other preparation. The composition will be applied at various stages including while the plant is still in the ground; post-harvest; and prior to, during, or after shipping. Application of the composition will reduce post-harvest infection and contamination by plant, animal, and human pathogens.

“Effective amount” refers to an amount of a compound that can be therapeutically effective to inhibit, prevent or treat the symptoms of particular disease, disorder, or side effect, particularly those associated with bacterial virulence. An effective amount of the active compound will be used alone or as part of a composition as described herein. In various embodiments, an effective amount of active compound in the composition will be from about 0.1 μg/g to about 0.9 g/g, about 1 μg/g to about 100 mg/g, about 10 μg/g to about 10 mg/g, and about 100 μg/g to about 1 mg/g. The amount of active compound applied to a surface (e.g. soil, stem, or a leaf) will be about 0.1 μg/sq.ft., about 1 μg/sq.ft., about 10 μg/sq.ft., about 100 μg/sq.ft., about 1 mg/sq.ft., about 10 mg/sq.ft., about 100 mg/sq.ft., about 1 g/sq.ft., about 10 g/sq.ft., or about 100 g/sq.ft.

The composition will be applied once or in repeated applications. Applications will be repeated any number of times daily, weekly, monthly, or annually. Applications will be repeated about 1 to about 100 times per day, week, month, or year, as needed.

Applying a composition comprising an active compound will reduce virulence of bacteria associated with the plant.

### Example 13

### Use with Animals Including Humans

The active compound or pharmaceutical composition comprising the active compound will be administered to an animal subject by any convenient route of administration, whether systemically/peripherally or at the site of desired action, including but not limited to, oral (e.g. by ingestion); topical (including e.g. transdermal, intranasal, ocular, buccal, and sublingual); pulmonary (e.g. by inhalation or insufflation therapy using, e.g. an aerosol, e.g. through mouth or nose); rectal; vaginal; parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot, for example, subcutaneously or intramuscularly. Additional modes of administration will include adding the active compound and/or a composition comprising the active compound to a food or beverage, including a water supply for an animal, to supply the active compound as part of the animal's diet.

The subject will include, without limitation, a eukaryote, an animal, a vertebrate animal, a bird, a reptile, an insect, a mammal, a rodent (e.g. a guinea pig, a hamster, a rat, a mouse), murine (e.g. a mouse), canine (e.g. a dog), feline (e.g. a cat), equine (e.g. a horse), an ovine (e.g. a sheep), a bovine, a primate, simian (e.g. a monkey or ape), a monkey (e.g. marmoset, baboon), an ape (e.g. gorilla, chimpanzee, orangutan, gibbon), or a human.

While it is possible for the active compound to be administered alone, in some embodiments the active compound will be presented as a pharmaceutical composition (e.g., formulation) comprising at least one active compound, as defined above, together with one or more pharmaceutically-acceptable carriers, adjuvants, excipients, diluents, fillers, buffers, stabilizers, preservatives, lubricants, or other materials well known to those skilled in the art and optionally other therapeutic or prophylactic agents.

Thus, the present invention further provides pharmaceutical compositions, as defined above, and methods of making a pharmaceutical composition comprising admixing at least one active compound, as defined above, together with one or more pharmaceutically acceptable carriers, excipients, buffers, adjuvants, stabilizers, or other materials, as described herein.

The term “pharmaceutically acceptable” as used herein pertains to compounds, materials, compositions, and/or dosage forms which are, within the scope of sound medical judgment, suitable for use in contact with the tissues of a subject (e.g., human or other animal) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Suitable carriers, excipients, etc. can be found in standard pharmaceutical texts, for example Remington's Pharmaceutical Sciences, 18th edition, Mack Publishing Company, Easton, Pa., 1990.

The formulations will conveniently be presented in unit dosage form and will be prepared by any method well known in the art of pharmacy. Such methods include the step of bringing into association the active compound with the carrier which constitutes one or more accessory ingredients. In general, the formulations will be prepared by uniformly and intimately bringing into association the active compound with liquid carriers or finely divided solid carriers or both, and then if necessary shaping the product.

Formulations may be in the form of liquids, solutions, suspensions, emulsions, elixirs, syrups, tablets, lozenges, granules, powders, capsules, cachets, pills, ampoules, suppositories, pessaries, ointments, gels, pastes, creams, sprays, mists, foams, lotions, oils, boluses, electuaries, or aerosols.

Formulations suitable for oral administration (e.g., by ingestion) may be presented as discrete units such as capsules, cachets or tablets, each containing a predetermined amount of the active compound; as a powder or granules; as a solution or suspension in an aqueous or non-aqueous liquid; or as an oil-in-water liquid emulsion or a water-in-oil liquid emulsion; as a bolus; as an electuary; or as a paste.

A tablet may be made by conventional means, e.g., compression or molding, optionally with one or more accessory ingredients. Compressed tablets may be prepared by compressing in a suitable machine the active compound in a free-flowing form such as a powder or granules, optionally mixed with one or more binders (e.g., povidone, gelatin, acacia, sorbitol, tragacanth, hydroxypropylmethyl cellulose); fillers or diluents (e.g., lactose, microcrystalline cellulose, calcium hydrogen phosphate); lubricants (e.g., magnesium stearate, talc, silica); disintegrants (e.g., sodium starch glycolate, cross-linked povidone, cross-linked sodium carboxymethyl cellulose); surface-active or dispersing or wetting agents (e.g., sodium lauryl sulfate); and preservatives (e.g., methyl p-hydroxybenzoate, propyl p-hydroxybenzoate, sorbic acid). Molded tablets may be made by molding in a suitable machine a mixture of the powdered compound moistened with an inert liquid diluent. The tablets may optionally be coated or scored and may be formulated so as to provide slow or controlled release of the active compound therein using, for example, hydroxypropylmethyl cellulose in varying proportions to provide the desired release profile. Tablets may optionally be provided with an enteric coating, to provide release in parts of the gut other than the stomach.

Formulations suitable for topical administration (e.g., transdermal, intranasal, ocular, buccal, and sublingual) may be formulated as an ointment, cream, suspension, lotion, powder, solution, past, gel, spray, aerosol, or oil. Alternatively, a formulation may comprise a patch or a dressing such as a bandage or adhesive plaster impregnated with active compounds and optionally one or more excipients or diluents. In addition, a formulation may be added to a conventional bandage, e.g. to a gauze portion that contacts the wound, as an antimicrobial agent.

Formulations suitable for topical administration in the mouth include losenges comprising the active compound in a flavored basis, usually sucrose and acacia or tragacanth; pastilles comprising the active compound in an inert basis such as gelatin and glycerin, or sucrose and acacia; and mouthwashes comprising the active compound in a suitable liquid carrier.

Formulations suitable for topical administration to the eye also include eye drops wherein the active compound is dissolved or suspended in a suitable carrier, especially an aqueous solvent for the active compound.

Formulations suitable for nasal administration, wherein the carrier is a solid, include a coarse powder having a particle size, for example, in the range of about 20 to about 500 microns which is administered in the manner in which snuff is taken, i.e., by rapid inhalation through the nasal passage from a container of the powder held close up to the nose. Suitable formulations wherein the carrier is a liquid for administration as, for example, nasal spray, nasal drops, or by aerosol administration by nebulizer, include aqueous or oily solutions of the active compound.

Formulations suitable for administration by inhalation include those presented as an aerosol spray from a pressurized pack, with the use of a suitable propellant, such as dichlorodifluoromethane, trichlorofluoromethane, dichoro-tetrafluoroethane, carbon dioxide, or other suitable gases.

Formulations suitable for topical administration via the skin include ointments, creams, and emulsions. When formulated in an ointment, the active compound may optionally be employed with either a paraffinic or a water-miscible ointment base. Alternatively, the active compounds may be formulated in a cream with an oil-in-water cream base. If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the active compound through the skin or other affected areas. Examples of such dermal penetration enhancers include dimethylsulfoxide and related analogues.

When formulated as a topical emulsion, the oily phase may optionally comprise merely an emulsifier (otherwise known as an emulgent), or it may comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and/or fat make up the so-called emulsifying ointment base which forms the oily dispersed phase of the cream formulations.

Suitable emulgents and emulsion stabilizers include Tween 60, Span 80, cetostearyl alcohol, myristyl alcohol, glyceryl monostearate and sodium lauryl sulphate. The choice of suitable oils or fats for the formulation is based on achieving the desired cosmetic properties, since the solubility of the active compound in most oils likely to be used in pharmaceutical emulsion formulations may be very low. Thus the cream should preferably be a non-greasy, non-staining and washable product with suitable consistency to avoid leakage from tubes or other containers. Straight or branched chain, mono- or dibasic alkyl esters such as diisoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for rectal administration may be presented as a suppository with a suitable base comprising, for example, cocoa butter or a salicylate.

Formulations suitable for vaginal administration may be presented as pessaries, tampons, creams, gels, pastes, foams or spray formulations containing in addition to the active compound, such carriers as are known in the art to be appropriate.

Formulations suitable for parenteral administration (e.g., by injection, including cutaneous, subcutaneous, intramuscular, intravenous and intradermal), include aqueous and non-aqueous isotonic, pyrogen-free, sterile injection solutions which may contain anti-oxidants, buffers, preservatives, stabilizers, bacteriostats in addition to the active compound, and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. Examples of suitable isotonic vehicles for use in such formulations include Sodium Chloride Injection, Ringer's Solution, or Lactated Ringer's Injection. Typically, the concentration of the active compound in the solution is from about 1 ng/ml to about 1 μg/ml, although other concentrations are possible and are encompassed within the invention. The formulations may be presented in unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets. Formulations may be in the form of liposomes or other microparticulate systems which are designed to target the active compound to blood components or one or more organs.

It will be appreciated that appropriate dosages of the active compounds, and compositions comprising the active compounds, can vary from patient to patient. Determining the optimal dosage will generally involve the balancing of the level of therapeutic benefit against any risk or deleterious side effects of the treatments of the present invention. The selected dosage level will depend on a variety of factors including, but not limited to, the activity of the particular compound, the route of administration, the time of administration, the rate of excretion of the compound, the duration of the treatment, other drugs, compounds, and/or materials used in combination, and the age, sex, weight, condition, general health, and prior medical history of the patient. The amount of compound and route of administration will ultimately be at the discretion of the physician, although generally the dosage will be to achieve local concentrations at the site of action which achieve the desired effect without causing substantial harmful or deleterious side-effects.

Administration in vivo can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

In general, a suitable dose of the active compound is in the range of about 100 μg to about 250 mg per kilogram body weight of the subject per day. Where the active compound is a salt, an ester, prodrug, or the like, the amount administered is calculated on the basis of the parent compound and so the actual weight to be used is increased proportionately.

Further information regarding formulation of and treatment with pharmaceutical compositions are disclosed in U.S. Pat. No. 7,196,085, incorporated herein by reference.

“Effective amount” refers to an amount of a compound that can be therapeutically effective to inhibit, prevent or treat the symptoms of particular disease, disorder, or side effect, particularly those associated with bacterial virulence. Thus, an effective amount of the active compound will be used alone or as part of a composition as described herein to treat an animal subject having a bacterium associated therewith in order to reduce the virulence of the bacterium. The composition will be prepared as appropriate for oral, topical, pulmonary, parenteral, or other route of administration. The concentration of active compound in the composition will be from about 0.1 μg/g to about 0.9 g/g, about 1 μg/g to about 100 mg/g, about 10 μg/g to about 10 mg/g, and about 100 μg/g to about 1 mg/g. When administered internally (e.g. parenterally or orally), the dose to the subject will be about 0.1 μg/kg body weight to about 1.0 g/kg body weight, about 1 μg/kg body weight to about 100 mg/kg body weight, about 10 μg/kg body weight to about 10 mg/kg body weight, and about 100 μg/kg body weight to about 1 mg/kg body weight.

The composition will be administered once, on a continuous basis (e.g. by an intravenous drip), or on a periodic/intermittent basis, including about once per hour, about once per two hours, about once per four hours, about once per eight hours, about once per twelve hours, about once per day, about once per two days, about once per three days, about twice per week, about once per week, and about once per month. The composition will be administered until a desired reduction of symptoms is achieved, which may be taken as an indication that bacterial virulence has been reduced.

Treatment of an animal subject with an active compound, or a composition comprising an active compound, will reduce virulence of bacteria associated with the animal subject.

### Example 14

### Use on Surfaces

In one embodiment, the active compounds selected from the phenylpropanoid derivatives will be applied to surfaces to reduce the virulence of bacteria on the surfaces. The active compounds will be formulated in compositions suitable for application to surfaces, including as a gel, powder, liquid, concentrate, spray, or other suitable compositions known to those skilled in the art.

The composition will be applied to surfaces in residential, commercial, medical, industrial, agricultural, and other settings, to reduce virulence of bacteria associated with the surfaces. The composition will be applied to surfaces including without limitation those in bathrooms; kitchens and other food storing or preparation areas; medical facilities including operating rooms and hospital rooms; laundry facilities including laundered articles; restaurant facilities including kitchens and food storage areas; refrigerators and freezers; dishwashing facilities; factories; slaughterhouses; and grocery stores. The active compounds will be incorporated into or applied on packaging, e.g. for medical items or food products, to reduce virulence of bacteria associated therewith.

In one embodiment, the active compound and/or a composition comprising the active compound will be used to reduce or inhibit biofilm formation in medical, industrial, and other equipment, where a biofilm is defined as an aggregation of microorganisms on a solid substrate. The active compound will be applied to external and internal surfaces of the equipment, including pipes and tubing, to reduce or inhibit bacterial virulence and biofilm formation.

“Effective amount” refers to an amount of the active compound that can be effective to reduce virulence of a bacterium associated with a surface when the compound, or a composition comprising the compound, is administered to a surface that includes the bacterium. An effective amount of the active compound will be used alone or as part of a composition as described herein. In various embodiments, an effective amount of the active compound in the composition will be from about 0.1 μg/g to about 0.9 g/g, about 1 μg/g to about 100 mg/g, about 10 μg/g to about 10 mg/g, and about 100 μg/g to about 1 mg/g. The amount of active compound applied to a surface will be about 0.1 μg/sq.ft., about 1 μg/sq.ft., about 10 μg/sq.ft., about 100 μg/sq.ft., about 1 mg/sq.ft., about 10 mg/sq.ft., about 100 mg/sq.ft., about 1 g/sq.ft., about 10 g/sq.ft., or about 100 g/sq.ft.

The composition will be sprayed, dusted, spread, rubbed, painted, mopped, soaked, or otherwise applied on surfaces. The composition will be applied once or will be repeated on a periodic basis. Periodic application will be about 1 to about 100 times per day, week, month, or year, as needed.

Applying a composition to a surface comprising an active compound will reduce virulence of bacteria on or associated with the surface.

Throughout this disclosure, various aspects of this invention may be presented in a range format. It should be understood that the description in range format is merely for convenience and brevity, and should not be construed as an inflexible limitation on the scope of the invention. Accordingly, as will be understood by one skilled in the art, for any and all purposes, particularly in terms of providing a written description, all ranges disclosed herein also encompass any and all possible subranges and combinations of subranges thereof, as well as integral and fractional numerical values within that range.

The above detailed description of the invention is illustrative of certain embodiments of the invention and is not intended to limit the scope of the invention as set forth in the appended claims.

1. A method of reducing virulence in a bacterium comprising at least one of a GacS/GacA-type system, a HrpX/HrpY-type system, a T3SS-type system, and a Rsm-type system, the method comprising contacting the bacterium with an effective amount of a phenylpropanoid-type inhibitory compound.

2. The method of claim 1, wherein the phenylpropanoid-type inhibitory compound is a compound of formula (II): 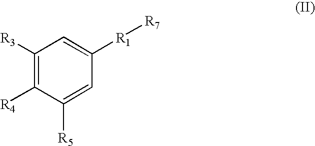
wherein R₁ is an alkylene;
R₃ and R₅ are hydrogen, R₄ is hydrogen, hydroxy, sulfhydryl or halo, and R₇ is hydroxy, carboxy or formyl;
R₃, R₄, and R₅ are independently selected from hydrogen, alkyl, alkenyl, alkynyl, cycloalkyl, heterocyclyl, aryl, heteroaryl, halo, hydroxy, ether, alkoxy, acetal, hemiacetal, ketal, hemiketal, formyl, acyl, carboxy, thiocarboxy, thiolcarboxy, thionocarboxy, imidic acid, hydroxamic acid, ester, acyloxy, oxycarboyloxy, amino, amido, thioamido, acylamido, aminocarbonyloxy, ureido, guanidine, amindino, nitro, nitroso, azido, cyano, isocyano, isocyanato, thiocyano, isothiocyano, sulfhydryl, thioether, disulfide, sulfine, sulfonyl, sulfinic acid, sulfonic acid, sulfinate, sulfonate, sulfinyloxy, sulfonyloxy, sulfate, sulfamyl, sulfonamide, sulfamino, sulfonamino, sulfinamino, phosphino, phosphor, phosphinyl, phosphono, phosphonate, phosphonooxy, phosphate, phosphorous acid, phosphite, phosphoramidite, and phosphoramidate, wherein two of R₃, R₄, and R₅ optionally are linked together to form a ring; and
R₇ is hydroxy, acetal, hemiacetal, ketal, hemiketal, formyl, acyl, carboxy, thiocarboxy, thiolcarboxy, thionocarboxy, imidic acid, hydroxamic acid, ester, acyloxy, oxycarboyloxy, amino, amido, thioamido, acylamido, aminocarbonyloxy, ureido, guanidine, amindino, nitro, nitroso, azido, cyano, isocyano, isocyanato, thiocyano, isothiocyano, sulfhydryl, thioether, disulfide, sulfine, sulfonyl, sulfinic acid, sulfonic acid, sulfinate, sulfonate, sulfinyloxy, sulfonyloxy, sulfate, sulfamyl, sulfonamide, sulfamino, sulfonamino, sulfinamino, phosphino, phosphor, phosphinyl, phosphono, phosphonate, phosphonooxy, phosphate, phosphorous acid, phosphite, phosphoramidite, or phosphoramidate.

3. The method of claim 1, wherein the phenylpropanoid-type inhibitory compound is p-coumaric acid or cinnamyl alcohol.

4. The method of claim 1, wherein the bacterium is Gram negative.

5. The method of claim 1, wherein the bacterium is a member of the Enterobacteriaceae family.

6. The method of claim 1, wherein the bacterium is of a bacterial genus selected from the group consisting of Pseudomonas, Erwinia, Azotobacter, Vibrio, Yersinia, Pectobacterium, Salmonella, and Escherichia.

7-10. (canceled)

11. The method of claim 1, wherein the bacterium is associated with a subject, wherein the bacteria is contacted with the phenylpropanoid-type compound by administering the compound to the subject.

12-13. (canceled)

14. The method of claim 11, wherein the subject is a plant, an animal or a human.

15. The method of claim 14, wherein the subject is a plant.

16. The method of claim 15, wherein the composition is administered via water, via soil, or topically.

17. The method of claim 11, wherein the composition is administered at least daily, weekly, monthly, or annually.

18. The method of claim 15, wherein the composition is sprayed on the plant.

19. The method of claim 11, wherein the subject is an animal and the composition further comprises a pharmaceutically-acceptable carrier or diluent.

20. The method of claim 19, wherein the subject is a human.

21. The method of claim 11, wherein the composition is administered topically, orally, or parenterally.

22-27. (canceled)

28. The method of claim 1, wherein the bacterium is on a surface and wherein the bacterium is contacted with the compound by contacting the surface with the compound.

29-30. (canceled)

31. The method of claim 28, wherein the surface is in a medical, industrial, commercial, or residential setting.

32-37. (canceled)

38. A pharmaceutical composition comprising a phenylpropanoid-type inhibitory compound according to formula (II) and a pharmaceutically-acceptable carrier or diluent: 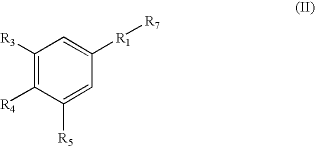
wherein R₁ is an alkylene;
R₃ and R₅ are hydrogen, R₄ is hydrogen, hydroxy, sulfhydryl or halo, and R₇ is hydroxy, carboxy or formyl;
R₃, R₄, and R₅ are independently selected from hydrogen, alkyl, alkenyl, alkynyl, cycloalkyl, heterocyclyl, aryl, heteroaryl, halo, hydroxy, ether, alkoxy, acetal, hemiacetal, ketal, hemiketal, formyl, acyl, carboxy, thiocarboxy, thiolcarboxy, thionocarboxy, imidic acid, hydroxamic acid, ester, acyloxy, oxycarboyloxy, amino, amido, thioamido, acylamido, aminocarbonyloxy, ureido, guanidine, amindino, nitro, nitroso, azido, cyano, isocyano, isocyanato, thiocyano, isothiocyano, sulfhydryl, thioether, disulfide, sulfine, sulfonyl, sulfinic acid, sulfonic acid, sulfinate, sulfonate, sulfinyloxy, sulfonyloxy, sulfate, sulfamyl, sulfonamide, sulfamino, sulfonamino, sulfinamino, phosphino, phosphor, phosphinyl, phosphono, phosphonate, phosphonooxy, phosphate, phosphorous acid, phosphite, phosphoramidite, and phosphoramidate, wherein two of R₃, R₄, and R₅ optionally are linked together to form a ring; and
R₇ is hydroxy, acetal, hemiacetal, ketal, hemiketal, formyl, acyl, carboxy, thiocarboxy, thiolcarboxy, thionocarboxy, imidic acid, hydroxamic acid, ester, acyloxy, oxycarboyloxy, amino, amido, thioamido, acylamido, aminocarbonyloxy, ureido, guanidine, amindino, nitro, nitroso, azido, cyano, isocyano, isocyanato, thiocyano, isothiocyano, sulfhydryl, thioether, disulfide, sulfine, sulfonyl, sulfinic acid, sulfonic acid, sulfinate, sulfonate, sulfinyloxy, sulfonyloxy, sulfate, sulfamyl, sulfonamide, sulfamino, sulfonamino, sulfinamino, phosphino, phosphor, phosphinyl, phosphono, phosphonate, phosphonooxy, phosphate, phosphorous acid, phosphite, phosphoramidite, or phosphoramidate.

39. The method of claim 38, wherein the phenylpropanoid-type inhibitory compound is p-coumaric acid or cinnamyl alcohol.

40. A method of screening a compound for an ability to reduce virulence of a bacterium comprising at least one of a GacS/GacA-type system, a HrpX/HrpY-type system, a T3SS-type system, and a Rsm-type system, the method comprising:
contacting the bacterium with a phenylpropanoid derivative; and
detecting at least one of: (i) a change in a component of at least one of the GacS/GacA-type system, the HrpX/HrpY-type system, the T3SS-type system, and the Rsm-type system of the bacterium, and (ii) a change in host pathology.

41. The method of claim 40, wherein a phenylpropanoid derivative is a compound of formula (I): 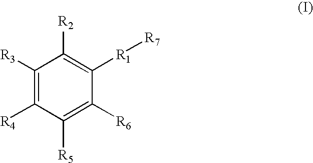
wherein R₁ is an alkylene;
R₂, R₃, R₄, R₅ and R₆ are independently selected from hydrogen, alkyl, alkenyl, alkynyl, cycloalkyl, heterocyclyl, aryl, heteroaryl, halo, hydroxy, ether, alkoxy, acetal, hemiacetal, ketal, hemiketal, formyl, acyl, carboxy, thiocarboxy, thiolcarboxy, thionocarboxy, imidic acid, hydroxamic acid, ester, acyloxy, oxycarboyloxy, amino, amido, thioamido, acylamido, aminocarbonyloxy, ureido, guanidine, amindino, nitro, nitroso, azido, cyano, isocyano, isocyanato, thiocyano, isothiocyano, sulfhydryl, thioether, disulfide, sulfine, sulfonyl, sulfinic acid, sulfonic acid, sulfinate, sulfonate, sulfinyloxy, sulfonyloxy, sulfate, sulfamyl, sulfonamide, sulfamino, sulfonamino, sulfinamino, phosphino, phosphor, phosphinyl, phosphono, phosphonate, phosphonooxy, phosphate, phosphorous acid, phosphite, phosphoramidite, and phosphoramidate, wherein two of R₂, R₃, R₄, R₅, and R₆ optionally are linked together to form a ring; and
R₇ is hydroxy, acetal, hemiacetal, ketal, hemiketal, formyl, acyl, carboxy, thiocarboxy, thiolcarboxy, thionocarboxy, imidic acid, hydroxamic acid, ester, acyloxy, oxycarboyloxy, amino, amido, thioamido, acylamido, aminocarbonyloxy, ureido, guanidine, amindino, nitro, nitroso, azido, cyano, isocyano, isocyanato, thiocyano, isothiocyano, sulfhydryl, thioether, disulfide, sulfine, sulfonyl, sulfinic acid, sulfonic acid, sulfinate, sulfonate, sulfinyloxy, sulfonyloxy, sulfate, sulfamyl, sulfonamide, sulfamino, sulfonamino, sulfinamino, phosphino, phosphor, phosphinyl, phosphono, phosphonate, phosphonooxy, phosphate, phosphorous acid, phosphite, phosphoramidite, or phosphoramidate.

42. The method of claim 40, wherein the component is a polynucleotide or a polypeptide and wherein detecting a change comprises measuring a level of expression of the polynucleotide or the polypeptide, or measuring an activity of the polypeptide.

43. The method of claim 40, wherein the component is a regulator of at least one of the GacS/GacA-type system, the HrpX/HrpY-type system, the T3SS-type system, and the Rsm-type system.

44. The method of claim 40, wherein the polynucleotide comprises at least one of gacA, hrpS, hrpL, dspE, hrpA, hrpN, rsmA, rsmB, rsmC, pel, pelD, pelL, hrpY, and hrpX.

45. The method claim 40, wherein the polypeptide is PelD, PelL, pectate lyase (Pel), protease (Prt), or cellulase (Cel).

46. The method of claim 40, wherein the component is associated with virulence of the bacterium.

47. The method of claim 46, wherein the component comprises pectinase, exoprotease, syringomycin, syringolin, alginate, tolaasin, siderophores, pyocyanin, cyanide, lipase, cholera toxin, or polyhydroxybutyrate.

48. The method of claim 40, wherein the component is an effector of the T3SS-type system.

49. The method of claim 40, wherein the component is a repressor of the T3SS-type system.

50. The method of claim 40, wherein the bacterium is pathogenic for a eukaryotic organism.

51. (canceled)

52. The method of claim 40, wherein the bacterium is Gram negative.

53. The method of claim 40, wherein the bacterium is a member of the Enterobacteriaceae family.

54. The method of claim 40, wherein the bacterium is of a bacterial genus selected from the group consisting of Pseudomonas, Erwinia, Azotobacter, Vibrio, Yersinia, Pectobacterium, Salmonella, and Escherichia.

55. The method of claim 40, wherein the bacterium is a Pseudomonas spp. selected from the group consisting of P. aureofaciens, P. chlororaphis, P. fluorescens, P. marginalis, P. syringae, P. tolaasii, P. viridiflava, and P. aeruginosa.

56. The method of claim 40, wherein the bacterium is an Erwinia-related strain selected from the group consisting of Dickeya dadantii, Erwinia carotovora, Erwinia atroseptica, and Erwinia amylovora.

57. The method of claim 40, wherein the bacterium is a Salmonella spp. selected from the group consisting of S. typhimurium and S. enterica.

58. The method of claim 43, wherein the change is a posttranslational modification of the regulator.

59. The method of claim 58, wherein the regulator is HrpL, HrpY, GacA, GacS, HrpS, HrpL, or a homolog thereof.

60. The method of claim 42, wherein polynucleotide is an mRNA.

61. The method of claim 42, wherein measuring the polynucleotide comprises a marker operably linked to a promoter.

62. The method of claim 40, wherein detecting the change in the component comprises conducting at least one of a promoter-probe bioreporter assay, a pectinase activity assay, and a qRT-PCR analysis.


SEQUENCE LISTING (36 sequences)

SEQ ID NO: 1 - DNA, 18 bp, Artificial Sequence
  Synthetic Oligonucleotide phrpA_F
gtgccgatagccagtgat

SEQ ID NO: 2 - DNA, 18 bp, Artificial Sequence
  Synthetic Oligonucleotide phrpA_R
tgctgctgcgttagaaag

SEQ ID NO: 3 - DNA, 20 bp, Artificial Sequence
  Synthetic Oligonucleotide phrpS_F
cagattgtatttgcggattg

SEQ ID NO: 4 - DNA, 22 bp, Artificial Sequence
  Synthetic Oligonucleotide phrpS_R
cggattcattgctattccttat

SEQ ID NO: 5 - DNA, 24 bp, Artificial Sequence
  Synthetic Oligonucleotide rplU_RTF
gcggcaaaatcaaggctgaagtcg

SEQ ID NO: 6 - DNA, 24 bp, Artificial Sequence
  Synthetic Oligonucleotide rplU_RTR
cggtggccagcctgcttacggtag

SEQ ID NO: 7 - DNA, 19 bp, Artificial Sequence
  Synthetic Oligonucleotide hrpY_RTF
cggcgacgggcgtaatgaa

SEQ ID NO: 8 - DNA, 20 bp, Artificial Sequence
  Synthetic Oligonucleotide hrpY_RTR
tttcggcgatggcattgacc

SEQ ID NO: 9 - DNA, 20 bp, Artificial Sequence
  Synthetic Oligonucleotide hrpS_RTF
tggaaggcgaaaccggcacc

SEQ ID NO: 10 - DNA, 19 bp, Artificial Sequence
  Synthetic Oligonucleotide hrpS_RTR
gcacggcggcgcagttcac

SEQ ID NO: 11 - DNA, 24 bp, Artificial Sequence
  Synthetic Oligonucleotide hrpL_RTF
gatgatgctgctggatgccgatgt

SEQ ID NO: 12 - DNA, 24 bp, Artificial Sequence
  Synthetic Oligonucleotide hrpL_RTR
tgcatcaacagcctggcggagata

SEQ ID NO: 13 - DNA, 20 bp, Artificial Sequence
  Synthetic Oligonucleotide hrpA_RTF
cagcaatggcaggcatgcag

SEQ ID NO: 14 - DNA, 20 bp, Artificial Sequence
  Synthetic Oligonucleotide hrpA_RTR
ctggccgtcggtgattgagc

SEQ ID NO: 15 - DNA, 22 bp, Artificial Sequence
  Synthetic Oligonucleotide dspE_RTF
gatggcggagctgaaatcgttc

SEQ ID NO: 16 - DNA, 22 bp, Artificial Sequence
  Synthetic Oligonucleotide dspE_RTR
ccttgccggaccgcttatcatt

SEQ ID NO: 17 - DNA, 24 bp, Artificial Sequence
  Synthetic Oligonucleotide rsmB_RTF
agagggatcgccagcaaggattgt

SEQ ID NO: 18 - DNA, 22 bp, Artificial Sequence
  Synthetic Oligonucleotide rsmB_RTR
cgtttgcagcagtcccgctacc

SEQ ID NO: 19 - DNA, 21 bp, Artificial Sequence
  Synthetic Oligonucleotide gacA_A
gcacccgattgcctgtactta

SEQ ID NO: 20 - DNA, 22 bp, Artificial Sequence
  Synthetic Oligonucleotide gacA_B
gcaccagttcatggtcatcaac

SEQ ID NO: 21 - DNA, 22 bp, Artificial Sequence
  Synthetic Oligonucleotide gacA_C
cggagacattgattagtagtga

SEQ ID NO: 22 - DNA, 20 bp, Artificial Sequence
  Synthetic Oligonucleotide gacA_D
attgggaaacgggccgaagt

SEQ ID NO: 23 - DNA, 18 bp, Artificial Sequence
  Synthetic Oligonucleotide PpelL_F
atgcggtaatgcggggat

SEQ ID NO: 24 - DNA, 20 bp, Artificial Sequence
  Synthetic Oligonucleotide PpelL_R
ggccagaactgatgtactgt

SEQ ID NO: 25 - DNA, 19 bp, Artificial Sequence
  Synthetic Oligonucleotide gacAco_F
gccaatgtttcgggtgtag

SEQ ID NO: 26 - DNA, 21 bp, Artificial Sequence
  Synthetic Oligonucleotide gacAco_R
catcgatctgccggatacttt

SEQ ID NO: 27 - DNA, 24 bp, Artificial Sequence
  Synthetic Oligonucleotide RplUsF
gcggcaaaatcaaggctgaagtcg

SEQ ID NO: 28 - DNA, 24 bp, Artificial Sequence
  Synthetic Oligonucleotide RplUsR
cggtggccagcctgcttacggtag

SEQ ID NO: 29 - DNA, 24 bp, Artificial Sequence
  Synthetic Oligonucleotide HrpLsF
gatgatgctgctggatgccgatgt

SEQ ID NO: 30 - DNA, 24 bp, Artificial Sequence
  Synthetic Oligonucleotide HrpLsR
tgcatcaacagcctggcggagata

SEQ ID NO: 31 - DNA, 24 bp, Artificial Sequence
  Synthetic Oligonucleotide rsmAf
ttttgactcgtcgagttggcgaaa

SEQ ID NO: 32 - DNA, 24 bp, Artificial Sequence
  Synthetic Oligonucleotide rsmAr
gcgcgttaacaccgatacgaacct

SEQ ID NO: 33 - DNA, 24 bp, Artificial Sequence
  Synthetic Oligonucleotide rsmBf
agagggatcgccagcaaggattgt

SEQ ID NO: 34 - DNA, 22 bp, Artificial Sequence
  Synthetic Oligonucleotide rsmBr
cgtttgcagcagtcccgctacc

SEQ ID NO: 35 - DNA, 24 bp, Artificial Sequence
  Synthetic Oligonucleotide rsmCf
acgaagtgctcccggttaatgtcc

SEQ ID NO: 36 - DNA, 24 bp, Artificial Sequence
  Synthetic Oligonucleotide rsmCr
acgagagcgtactgagcggctttt